(19) United States
(12) Patent Application Publication
(10) Pub. No.: US 2020/0179327 A1
(43) Pub. Date: Jun. 11, 2020

(54) USE OF PHENOLIC COMPOUNDS FROM OLEA EUROPAEA

(71) Applicant: McCord Research, Inc., Coralville, IA (US)

(72) Inventors: Darlene E. McCord, Coralville, IA (US); Thomas Karagiannis, Melbourne (AU)

(21) Appl. No.: 16/638,277

(22) PCT Filed: Aug. 10, 2018
(86) PCT No.: PCT/US18/46288

§ 371 Date: Feb. 11, 2020

Related U.S. Application Data
(60) Provisional application No. 62/544,452, filed on Aug. 11, 2017.

(51) Int. Cl. A61K 31/351; A61K 31/222; A61P 29/00

(52) U.S. Cl. CPC A61K 31/351; A61K 36/63; A61P 29/00; A61K 31/222

(57) ABSTRACT

A series of phenolic compounds with similar inhibitory COX-1 and COX-2 properties as oleocanthal and oleuropein is described here. The phenolic compounds disclosed here were also found to be MAO and LSD-1 inhibitors. Also provided are methods of using these phenolic compounds in various formulations and compositions including food additives, pharmaceuticals, cosmetics, and animal repellants.

### CROSS-REFERENCE TO RELATED APPLICATIONS

This application claims priority and is related to U.S. Provisional Application Ser. 62/544,452 and entitled “COMPOUNDS FROM OLEA EUROPAEA.” The entire contents of this patent application are hereby expressly incorporated herein by reference including, without limitation, the specification, claims, and abstract, as well as any figures, tables, or drawings thereof.

### FIELD OF THE INVENTION

The present disclosure relates generally to a series of phenolic compounds derived from the compounds found in Olea europaea. In particular, the present disclosure relates to a series of phenolics compounds that are structurally similar to oleocanthal or oleuropein, which are found in olive oil and cause a strong stinging sensation in the throat. The disclosed compounds here are demonstrated to possess antioxidant, anti-inflammatory, anti-depression, and other chemotherapeutic activity that may contribute to the prevention of several chronic disease states, including but not limited to, depression and neurodegeneration.

### BACKGROUND OF THE INVENTION

The genus Olea (Oleaceae) contains approximately 40 taxa of evergreen shrubs and trees, found throughout southern Europe, Africa, Asia and Oceania. Native to the Mediterranean basin is O. europaea subsp. europaea, which is ubiquitously distributed throughout the region, and grown commercially for its fruit that contributes to the production of olive oil and table olives. Its hardiness and ubiquitous consumption have seen the cultivation of O. europaea endure from the Copper Age to the present day. O. europaea is now one of the most valuable crops worldwide.

Traditionally, the olive fruit and oil has been widely used in folk medicine, where it is used as a topical antiseptic as well as an analgesic for rheumatism and abdominal pain. However, in more recent times, the healthful effects associated with the consumption of the olive, has been exposed by studies encompassing the Mediterranean diet, where olive oil is the primary fat source.

Epidemiological and clinical studies have indicated that a low chronic disease risk within the Mediterranean region is in part due to the high intake of fruits and oils from the olive tree (Olea europaea L.). Historically, the health benefits of virgin olive oil (VOO) was attributed to the high level of oleic acid, a monounsaturated fatty acid (MUFA) (18:ln-9), which represents 55-83% of total fatty acids present in VOO. Although MUFAs have been shown to be beneficial to circulating lipids and lipoprotein profile, other seed oils (including sunflower, soybean and rapeseed) rich in MUFAs are largely ineffective in altering chronic disease risk factors. On the other hand, the minor component of VOO containing the phenolic fraction, has been shown to contribute to the cardioprotective effects of VOO.

Thus, it has been postulated that the MUFA content is not likely to be the main agent responsible for the health properties of VOO, but rather the minor bioactive constituents. It is now recognized that many of the beneficial effects of consuming olive-containing products is due to their unique minor compounds, including flavonoids, lignans, secoridoids, their hydrolysis products, and particularly phenolic compounds. These compounds have showcased a broad range of bioactive properties, including antioxidant, anti-inflammatory, and chemo-preventative activities which have been elucidated over the past 20 years.

However, the considerable diversity and complexity of the phenolic compounds associated with olive-containing products as well as the matrix in which they are found has posed a challenge to analytical chemists. As such, only a subset of olive phenolic compounds has been identified and explored by the pharmacological and biological sciences. Chiefly, tyrosol, hydroxytyrosol (HT), oleuropein and oleocanthal have been investigated for their biological activities. However, some other structurally similar compounds identified within the olive—some of which may not be commercially available, are yet to be validated by the scientific community.

Of particular interest to the olive research community and industry are the phenolic class of compounds, which contribute to the stability and organoleptic properties of the oil and convey important biological activities. Phenolic compounds encompass a diverse subset of chemical structures found within the leaf, fruit, and oil of the olive.

Although phenolic compounds can be simply characterized by an aromatic ring structure with one or more hydroxyl groups, they are difficult to characterize in terms of both structures, functions, and activities due to limitations of current analytical methodologies and lack of availability. So far, only oleocanthal and oleuropein are well studied and characterized. The structures of oleocanthal and oleuropein are shown below.

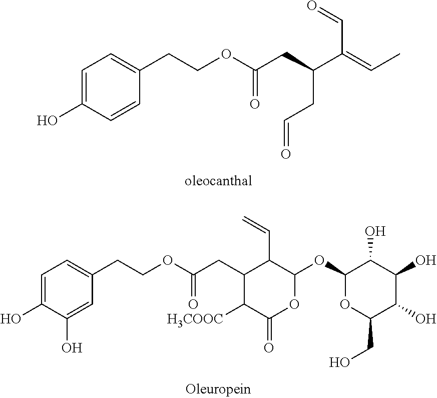

Oleocanthal was found to be a dose-dependent inhibitor of COX-1 and COX-2, but to have no effect on lipoxygenase in vitro, just like ibuprofen (Beauchamp, et al., Nature, V437, p 45-46, 2005). This founding raises the possibility that long-term consumption of oleocanthal may help protect against or mediate some diseases, such as Alzheimer's disease and those associated with inflammation.

U.S. Pat. No. 8,586,632 and US Patent Publication 2009/0076142 A1 disclose oleocanthal analogs and methods of using oleocanthal and some oleocanthal analogs in various formulations including food additives (e.g., flavor enhancers, sweetness inhibitors, spices, flavorings, and preservatives); pharmaceuticals (e.g., antioxidants, micro-G protein and associated kinase inhibitors, Aβ42 inhibitors, presenilin modifiers, γ-secretase inhibitors, nonsteroidal anti-inflammatories, anti-pyretics, cold and flu symptom relievers, COX-1, COX-2 inhibitors, COX-3 inhibitors, lipoxygenase inhibitors, and wound healers); cosmetics; animal repellants; and discovery tools for mammalian irritation receptor genes, gene products, alleles, splice variants, alternate transcripts, and the like.

Accordingly, it is an objective of the disclosure to identify other phenolic compounds derived from Olea europaea that have similar properties as oleocanthal has and to associate these phenolic compounds with their respective pharmacological activities.

It is also an objective of the disclosure to make use of the phenolic compounds disclosed herein for their pharmaceutic, health, cosmetic, and/or diet benefits.

It is a further objective of the disclosure to provide compositions or methods of using the disclosed phenolic compounds in various formulations, including food additives, pharmaceuticals, cosmetics, animal repellants, discovery tools for mammalian irritation receptor genes, gene products, alleles, splice variants, alternate transcripts, and the like.

### BRIEF SUMMARY OF THE INVENTION

In one aspect, provided here is a method of inhibiting COX-1 and/or COX-2 activities in a subject, wherein the method comprises administering an effective amount of a composition comprising compound having any one of the following formulas, stereo-isomer thereof, or salt thereof:

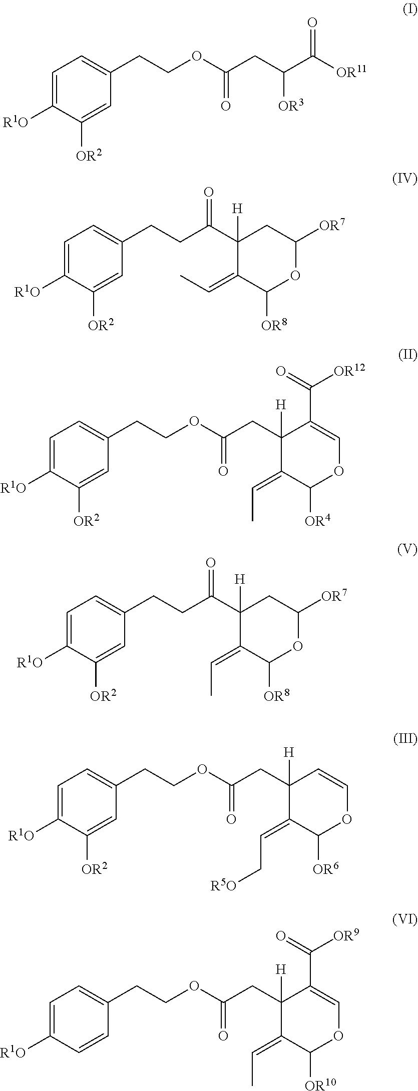

wherein R¹-R¹² are independently H or R²⁰; and R²⁰ is a H, C₁-C₅ alkyl, or a glycoside.

In another aspect, provided herein is a composition comprising a therapeutically effective amount of a compound having any of the formula I-VI, stereo-isomer thereof, or salt thereof and a pharmaceutically acceptable carrier.

In another aspect, provided herein is method of use comprising administering to a subject a composition comprising one or more compounds disclosed herein and above or their salts and a pharmaceutically acceptable carrier.

In yet another aspect, provided herein is a method of inhibiting MAO activity in a patient, wherein the method comprises administering to a patient an effective amount of a composition having one or more compounds disclosed herein and above or their salts and a pharmaceutically acceptable carrier.

In one aspect, provided herein is a method of inhibiting LSD-1 activity in a patient, wherein the method comprises administering an effective amount of a composition having one or more compounds disclosed herein and above or their salts and a pharmaceutically acceptable carrier.

In yet another aspect, provided herein is a method of treating a patient with an inflammatory disorder, wherein the method comprises administering to a patient with an inflammatory disorder an effective amount of a composition comprising a compound having any of the formula I-VI, stereo-isomer thereof, or salt thereof.

In one aspect, provided herein is a method of treating a patient with an neurodegenerative disorder, wherein the method comprises administering to a patient with an neurodegenerative disorder an effective amount of a composition comprising a compound having any of the formula I-VI, stereo-isomer thereof, or salt thereof.

In yet another aspect, provided herein is a method of treating a patient with a depression disorder, wherein the method comprises administering to a patient with a depression disorder an effective amount of a composition comprising a compound having any of the formula I-VI, stereo-isomer thereof, or salt thereof.

In yet another aspect, provided herein is a method of treating a patient with cancer, wherein the method comprises administering to a patient with cancer an effective amount of a composition comprising a compound having any of the formula I-VI, stereo-isomer thereof, or salt thereof.

In another aspect, provided here is an antioxidant composition comprising a therapeutically effective amount of a compound having any of the formula I-VI, stereo-isomer thereof, or salt thereof disclosed herein and a pharmaceutically acceptable carrier.

In another aspect, provided here is a method of enhancing the flavor of food comprising adding an effective amount of a compound having any of the formula I-VI, stereo-isomer thereof, or salt thereof disclosed herein.

In another further aspect, provided here is an animal repellent composition comprising an effective amount of a compound of a compound having any of the formula I-VI, stereo-isomer thereof, or salt thereof disclosed herein.

In another aspect, provided here is a method for treating a sore throat comprising administering to a patient with a sore throat an effective amount of a composition comprising a compound having any of the formula I-VI, stereo-isomer thereof, or salt thereof disclosed herein and a pharmaceutically acceptable carrier.

In yet another aspect, provided herein is a method of preserving food comprising contacting a food with an effective amount of a compound having any of the formula I-VI, stereo-isomer thereof, or salt thereof disclosed herein.

In another aspect, provided herein is a method of repelling animals from an edible source comprising adding to an edible source an effective amount of a compound having any of the formula I-VI, stereo-isomer thereof, or salt thereof disclosed herein.

In yet another aspect, provided herein is a method of inhibiting sweetness perception in an edible source comprising adding to an edible source a sweetness inhibiting amount of a compound having any of the formula I-VI, stereo-isomer thereof, or salt thereof disclosed herein.

In another aspect, provided herein is a method of treating a cold comprising administering to a patient in need of treatment a composition comprising an effective amount of a compound having any of the formula I-VI, stereo-isomer thereof, or salt thereof disclosed herein.

In yet another aspect, provided herein is a method of inhibiting growth of microorganisms comprising contacting microorganisms with an effective amount of a composition comprising a compound having any of the formula I-VI, stereo-isomer thereof, or salt thereof disclosed herein.

In another aspect, provided herein is a method of treating pain in a patient comprising administering to a patient an effective amount of a composition comprising a compound having any of the formula I-VI, stereo-isomer thereof, or salt thereof disclosed herein.

In yet another aspect, provided herein is a method of preventing a neurodegenerative disorder comprising administering to a patient an effective amount of a composition comprising a compound having any of the formula I-VI, stereo-isomer thereof, or salt thereof disclosed herein.

The significance of the disclosure herein is that the series of phenolic compounds found in Olea europaea was identified to have similar characteristic properties in terms of their interaction with COX-1 and/or COX-2 as other well-studied phenolic compounds. These compounds have a molecular weight of less than 500.

Although the health and therapeutic benefits of olive fruits, oil, and diets are well recognized, the minor components that contributes to these benefits have not been fully identified. The phenolic compounds disclosed herein were shown by data to be COX-1, COX-2, MAO-A, and LSD-1 inhibitor and can be therefore used as preventive and therapeutic agent for various disorders, such as neurodegenerative disorders, depression, and cancer or as cosmetic, food additive agent, etc_{[JS1]}.

The forgoing summary is illustrative only and is not intended to be in any way limiting. In addition to the illustrative aspects, embodiments, and features described above, further aspects, embodiments, and features of the present technology will become apparent to those skilled in the art from the following drawings and the detailed description, which shows and describes illustrative embodiments of the present technology. Accordingly, the figures and detailed description are also to be regarded as illustrative in nature and not in any way limiting.

### BRIEF DESCRIPTION OF THE DRAWING

FIG. 1 shows PSA vs. log P for the olive phenolics showing 95% and 99% confidence limits, denoted by ellipses corresponding to the blood-brain barrier and intestinal absorption models.

FIG. 2A-FIG. 2C show oleocanthal docked to COX-1 protein shown in 3-dimensional and 2-dimensional views. FIG. 2A shows the whole protein with oleocanthal;

FIG. 2B highlights oleocanthal in the binding pocket; FIG. 2C shows a 2D ligand interaction diagram between oleocanthal and residues of the COX-1 protein.

FIG. 3 shows the scheme for the synthesis of the exemplary phenolic compound (I-1), methyl malate-β-hydroxytyrosol ester (MMBHTE).

FIG. 4A, FIG. 4B, FIG. 4C, and FIG. 4D show the cell viability and proliferation of PBMC, BJ, h-HAEC, and DII-HAEC human primary cells lines treated with methyl malate-β-hydroxytyrosol ester, respectively.

FIG. 5 shows the inhibition profiles of MMBHTE, HT, and TCP for lysine-specific demethylase 1 (LSD1) enzyme activity.

FIG. 6A and FIG. 6B show the inhibition activities of MMBHTE and Oleocanthal on COX-1 and COX-2, respectively.

FIG. 7 shows the inhibition activities of MMBHTE, HT and TCP on monoamine oxidase A (MAO-A).

Various embodiments of the present disclosure will be described in detail with reference to the figures, wherein like reference numerals represent like parts throughout the several views. Reference to various embodiments does not limit the scope of the disclosure. Figures represented herein are not limitations to the various embodiments according to the disclosure and are presented for exemplary illustration of the disclosure.

### DETAILED DESCRIPTION OF THE INVENTION

The present disclosure relates to a series of phenolic compounds derived from oleo europaea. Through molecular docking and examination of pharmacophore arrangements of some common compounds that exhibited the strongest binding affinity to the putative inflammatory mediator cyclooxygenase (COX), a series of phenolic compounds was identified and disclosed in this disclosure. _{[JNL2]}These disclosed phenolic compounds were shown by experimental data to be effective inhibitors for COX-1, COX-2, MAO, and LSD-1 enzymes and can be used as preventive and therapeutic agent for various disorders, such as inflammatory, neurodegenerative, mental disorders, and cancer, for which olive fruits, oil and diets are found to be beneficial.

The embodiments of this disclosure are not limited to particular compositions and methods of use, which can vary and are understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g. 1 to 5 includes 1, 1.5, 2, 2.75, 3, 3.80, 4, and 5).

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts.

As used herein, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to carbon(s) or hydrogen(s) atom replaced by one or more bonds, including double or triple bonds, to a heteroatom. Thus, a substituted group is substituted with one or more substituents, unless otherwise specified. A substituted group can be substituted with 1, 2, 3, 4, 5, or 6 substituents.

Substituted ring groups include rings and ring systems in which a bond to a hydrogen atom is replaced with a bond to a carbon atom. Therefore, substituted cycloalkyl, aryl, heterocyclyl, and heteroaryl groups may also be substituted with substituted or unsubstituted alkyl, alkenyl, and alkynyl groups are defined herein.

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur, or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Alkenyl groups or alkenes are straight chain, branched, or cyclic alkyl groups having two to about 30 carbon atoms, and further including at least one double bond. In some embodiments, an alkenyl group has from 2 to about 20 carbon, or typically, from 2 to 10 carbon atoms. Alkenyl groups may be substituted or unsubstituted. For a double bond in a alkenyl group, the configuration for the double bond can be a trans or cis configuration. Alkenyl groups may be substituted similarly to alkyl groups.

Alkynyl groups are straight chain, branched, or cyclic alkyl groups having two to about 30 carbon atoms, and further including at least one triple bond. In some embodiments, an alkynyl group has from 2 to about 20 carbon, or typically, from 2 to 10 carbon atoms. Alkynyl groups may be substituted or unsubstituted. Alkynyl groups may be substituted similarly to alkyl or alkenyl groups.

As used herein, the terms “alkylene”, “cycloalkylene”, “alkynylene”, and “alkenylene”, alone or as part of another substituent, refer to a divalent radical derived from an alkyl, cycloalkyl, or alkenyl group, respectively, as exemplified by —CH₂CH₂CH₂—. For alkylene, cycloalkylene, alkynylene, and alkenylene groups, no orientation of the linking group is implied.

The term “ester” as used herein refers to —R³⁰COOR³¹ group. R³⁰ is absent, a substituted or unsubstituted alkylene, cycloalkylene, alkenylene, alkynylene, arylene, aralkylene, heterocyclylalkylene, or heterocyclylene group as defined herein. R³¹ is a substituted or unsubstituted alkyl, cycloalkyl, alkenyl, alkynyl, aryl, aralkyl, heterocyclylalkyl, or heterocyclyl group as defined herein.

The term “amine” (or “amino”) as used herein refers to —R³²NR³³R³⁴ groups. R³² is absent, a substituted or unsubstituted alkylene, cycloalkylene, alkenylene, alkynylene, arylene, aralkylene, heterocyclylalkylene, or heterocyclylene group as defined herein. R³³ and R³⁴ are independently hydrogen, or a substituted or unsubstituted alkyl, cycloalkyl, alkenyl, alkynyl, aryl, aralkyl, heterocyclylalkyl, or heterocyclyl group as defined herein.

The term “amine” as used herein also refers to an independent compound. When an amine is a compound, it can be represented by a formula of R^{32′}NR^{33′}R^{34′} groups, wherein R^{32′}, R^{33′}, and R³⁴ are independently hydrogen, or a substituted or unsubstituted alkyl, cycloalkyl, alkenyl, alkynyl, aryl, aralkyl, heterocyclylalkyl, or heterocyclyl group as defined herein.

The term “alcohol” as used herein refers to —R³⁵OH groups. R³⁵ is absent, a substituted or unsubstituted alkylene, cycloalkylene, alkenylene, alkynylene, arylene, aralkylene, heterocyclylalkylene, or heterocyclylene group as defined herein.

The term “carboxylic acid” as used herein refers to —R³⁶COOH groups. R³⁶ is absent, a substituted or unsubstituted alkylene, cycloalkylene, alkenylene, alkynylene, arylene, aralkylene, heterocyclylalkylene, or heterocyclylene group as defined herein.

The term “ether” as used herein refers to —R³⁷OR³⁸ groups. R³⁷ is absent, a substituted or unsubstituted alkylene, cycloalkylene, alkenylene, alkynylene, arylene, aralkylene, heterocyclylalkylene, or heterocyclylene group as defined herein. R³⁸ is a substituted or unsubstituted alkyl, cycloalkyl, alkenyl, alkynyl, aryl, aralkyl, heterocyclylalkyl, or heterocyclyl group as defined herein.

Uses of the phenolic compounds derived from Olea europaea Provided herein is a method of using a series of phenolic compounds that are either derived from Olea europaea or derivative thereof, just like oleocanthal or oleuropein. Since oleocanthal, oleuropein, or some of their derivatives are found to be a COX-1 and COX-2 inhibitor like ibuprofen, the disclosed phenolic compounds are structurally similar to oleocanthal and oleuropein. However, these phenolic compounds have not been specifically identified and attributed to any specific biological activities.

In addition, ibuprofen and oleocanthal were believed to inhibit micro-G proteins and associated kinases, for example Ras and Rock, which have been associated with the development of Aβ42 associated plaques and tangles in the brains of Alzheimer's patients. Oleocanthal also acts to inhibit γ-secretases and alter presenilin conformations of which both activities are associated with reducing Aβ42 associated Alzheimer's plaques and tangles.

It is also believed that certain non-steroidal anti-inflammatory drugs (NSAIDs) inhibit γ-secretases without significantly altering other activities in the Aβ amyloid precursor protein (APP) processing pathway. In patients with certain mutations in APP and all mutations known for presenilin, APP is processed such that there is a large increase in the amount of a proteolytic fragment of 40-42 residues (Aβ42).

Certain NSAIDs appear to have an effect of reducing the production of Aβ42 by a mechanism that is independent of the cyclooxygenase activity associated with the anti-inflammatory activity of the NSAIDs. It has been shown that for many NSAIDs, which are administered as racemic mixtures of the active compounds, that a specific enantiomer (the S-enantiomer) appears to be responsible for the inhibition of cyclooxygenase activity, and hence the anti-inflammatory effect.

It has also been shown that the R-enantiomer of the NSAIDs may mediate reduction of Aβ42 production and may be responsible for the decreased risk in Alzheimer's and cognitive impairment seen with long term use of NSAIDs.

Also correlating with lower risk of developing Alzheimer's and cognitive impairment is the so-called Mediterranean diet, which is typically high in consumption of, among other things, olive oil. Thus, the observation made herein of the association with the organoleptic properties of oleocanthal and the similarity to ibuprofen and the observations associated with long term use of NSAIDs and dietary intake of olive oil suggest that oleocanthal may be used for the treatment and prevention of neurodegenerative disorders. The treatment and prevention of such neurodegenerative disorders may be performed using a racemic mixture of oleocanthal, or one of the purified enantiomers of oleocanthal. Therefore, it is expected that the compounds disclosed herein can be used for the treatment and prevention of neurodegenerative disorders.

Neurodegenerative disorders, as used herein, refer to a range of conditions which primarily affect the neurons in the human brain. Neurons are the building blocks of the nervous system which includes the brain and spinal cord. Neurons normally don't reproduce or replace themselves, so when they become damaged or die they cannot be replaced by the body.

Some of the common symptoms of neurodegenerative disorders include, but not limited to, memory loss, forgetfulness, apathy, anxiety, agitation, loss of inhibition and mood changes.

Neurodegenerative disorders are currently incurable and debilitating conditions that result in progressive degeneration and/or death of nerve cells. This causes problems with movement (called ataxias), or mental functioning (called dementias). Dementias are responsible for the greatest burden of neurodegenerative orders.

Neurodegenerative disorders as referred herein include, but not limited to, Alzheimer's disease (AD) and other dementias; Parkinson's disease (PD) and PD-related disorders; prion disease; motor neuron diseases (MND); Huntington's disease (HD); spinocerebellar ataxia (SCA); and spinal muscular atrophy (SMA). Among these listed disorders, AD causes approximately 60-70% of dementia cases.

Since neurodegenerative disorders relates to one of four proteins that have gone rogue, tau, amyloid-beta (Aβ), alpha-synuclein (α-syn), and TDP-43, or a combination of several proteins, the phenolic compounds disclosed could have efficacy for preventing, slowing, or treating neurodegenerative disorders, since the phenolic compounds were found to behave and be structurally similar to oleocanthal or oleuropein. As such, investigational drugs and studies aimed at preventing or slowing the disease often hone in on just one of these respective proteins.

Mental disorders or mental illnesses are a large and diverse group of conditions that affect your behavior patterns. Some of the most frequently diagnosed mental disorders include, but not limited to, depression, panic disorder, social phobia, anxiety, bipolar disorder, post-traumatic stress disorder (PTSD), and schizophrenia.

The best-known anti-depressants are monoamine oxidase (MAO) inhibitors. MAO includes MAO-A and MAO-B. A MAO inhibitor can inhibit MAO-A, MAO-B, or both. The MAO inhibitors are also effective therapeutic agents for panic disorder and social phobia, and particularly effective in treatment-resistant depression and atypical depression. They are also used in the treatment of Parkinson's disease, one of the neurogenerative disorders.

Currently marketed MAO inhibitors include, for example, hydrazines such as isocarboxazid, nialamide, phenelzine, and hydracarbazine; non-hydrazines such as tranylcypromine; selective MAO-A inhibitors such as: bifemelane, moclobemide, pirlindole, and toloxatone; and selective MAO-B inhibitors such as rasagiline, selegiline, and safinamide; linezolid; and methylene blue.

One of drawbacks for MAO inhibitors is that they should not be combined with other psychoactive substances (antidepressants, painkillers, stimulants, both legal and illegal etc.) except under expert care. Certain combinations can cause lethal reactions. Common examples include selective serotonin reuptake inhibitors (SSRIs), tricyclics, 3,4-Methylenedioxymethamphetamine (MDMA, or ecstasy), meperidine, tramadol, and dextromethorphan. Drugs that affect the release or reuptake of epinephrine, norepinephrine, or dopamine typically need to be administered at lower doses due to the resulting potentiated and prolonged effect and MAO inhibitors can prevent metabolism of these drugs and cause their concentration to be much higher level than intended. MAO inhibitors also interact with tobacco-containing products (e.g., cigarettes) and may potentiate the effects of certain compounds in tobacco. This may be reflected in the difficulty of smoking cessation, as tobacco contains naturally occurring MAO inhibition compounds in addition to the nicotine.

List of substances with which MAO inhibitors _{[JNL3]}may adversely react include, but are not limited to, phenethlylamines (such as amphetamines), tryptamines, norepinephrine or dopamine or serotonin reuptake inhibitors (such as opioids), norepinephrine or dopamine or serotonin releasers (such as ephedrine), local and general anesthetic particularly those containing epinephrine, certain supplements such a St John's Wort, and certain anti-biotics.

Because of their toxicity or side effects, MAO inhibitors that have been withdrawn from the market include hydrazines such as: benmoxin, iproclozide, iproniazid, mebanazine, octamoxin, pheniprazine, phenoxypropazine, pivalylbenzhydrazine, and safrazine; non-hydrazine caroxazone; and selective MAO-A inhibitor minaprine.

Since the phenolic compounds disclosed here were found to be a better inhibitor than tranylcypromine (TCP, contracted from trans-2-phenylcyclopropylamine; original trade name Parnate) as a nonselective and irreversible inhibitor of the enzyme monoamine oxidase, they can be used as an antidepressant and anxiolytic agent in the clinical treatment of mood and anxiety disorders, respectively. Alternatively, since olive oil was not found to interfere _{[JNL4]}with selective serotonin reuptake inhibitors (SSRIs), tricyclics, 3,4-Methylenedioxymethamphetamine (MDMA, or ecstasy), meperidine, tramadol, and/or dextromethorphan, the various antidepressant therapies are suitable to be co-administered to a subject together with the phenolic compound(s) disclosed herein.

In addition, the phenolic compounds disclosed herein were also found to be better histone lysine-specific demethylase 1 (LSD-1_{[JNL5]}) inhibitors than tranylcypromine. LSD-1 is classified as a member of amine oxidase superfamily, the common feature of which is using the flavin adenine dinucleotide (FAD) as its cofactor. Since it is located in cell nucleus and acts as a histone methylation eraser, LSD-1 specifically removes mono- or dimethylated histone H3 lysine 4 (H3K4) and H3 lysine 9 (H3K9) through formaldehyde-generating oxidation. LSD1 and its downstream targets are involved in a wide range of biological courses, including embryonic development, tumor-cell growth, and metastasis. Since LSD-1 has been reported to be overexpressed in variety of tumors, inactivating LSD1 or downregulating its expression inhibits cancer-cell development. LSD1 targeting inhibitors may be new anticancer drugs.

Since the phenolic compounds disclosed here were found to be better inhibitors than tranylcypromine (TCP, contracted from trans-2-phenylcyclopropylamine; original trade name Parnate) as LSD-1 inhibitor, they can be used as anticancer drugs or agents for cancer prevention.

The other methods employing olive phenolics, such as Oleuropein (OL) and hydroxytyrosol (HT) and benefits thereof are described, for example, in U.S. patent application Ser. Nos. 14/328,843 and 14/731,441, which are incorporated herein in its entirety.

The phenolic compounds disclosed herein or their pharmaceutically acceptable salts or derivatives can be used as additives or preservatives in food for human or animal consumption, or consumer products. These compounds, their salts, or derivatives can be used as flavorants or flavor enhancers, such as an irritant to food for enhancing the flavor and gastronomic experience in a similar fashion to other spices such as chilis, mustards, onions, Szechwan pepper, and ginger. The phenolic compounds disclosed herein may be added to foods and oral pharmaceutical preparations and oral hygiene products such as toothpaste, mouthwash, breath-fresheners, films, candies, lozenges to provide an irritant for the oral product's sensory-irritation experience. Furthermore, the phenolic compounds disclosed herein may be added to health supplement products, as well. When the compounds disclosed herein used as food, product, supplement additives or preservatives, one or a mixture of one or more these compounds can be used, in pure or not so pure form.

Preferably, the phenolic compounds disclosed herein or their pharmaceutically acceptable salts or derivatives can be formulated with one or more pharmaceutically acceptable diluents, excipients, or carriers (collectively referred to herein as “carrier” materials) as one skilled in the art understands and practices to formulate any pharmaceutical compositions.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound which has been prepared by the addition of an acid. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds may exist as zwitterions. All forms of the compounds, including free acid, free base and zwitterions, are contemplated to be within the scope of the present disclosure.

The methods of the present disclosure comprise, consist of or consist essentially of administering at least one of the phenolic compounds or their salts disclosed herein or a pharmaceutical composition containing the phenolic compounds or their salts to a subject in need of a therapeutic or prophylactic treatment. As used herein, “administering” is meant a method of giving a dosage of at least one of the phenolic compounds or a pharmaceutical composition containing at least one of the phenolic compounds of the disclosure to an animal generally referred to as a “subject” or “patient,” both of which are herein understood to include human patients or a mammal. The compositions disclosed herein or utilized in the methods described herein can be administered by a route selected from, but are not limited to, parenteral, dermal, transdermal, ocular, inhalation, buccal, sublingual, perilingual, nasal, rectal, topical administration, and oral administration or ingestion. Parenteral administration includes intravenous, intraperitoneal, subcutaneous, intraarterial, intravascular, and intramuscular administration. The preferred method of administration can vary depending on various factors (e.g., the components of the composition being administered, and the severity of the condition being treated). In a preferred aspect, the administering is by ingestion, injection, infusion, or other bodily administration.

As one skilled in the art will ascertain, an amount sufficient to treat (e.g. therapeutically effective amount) refers to the amount of a pharmaceutical composition administered to improve, inhibit, or ameliorate a condition or a symptom of a disorder that is known to be associated with COX-1, COX-2, MAO-A, MAO-B, and/or LSD-1 protein activities of a subject, in a clinically relevant manner. Any improvement in the subject is sufficient to achieve treatment. Preferably, an amount sufficient to treat is an amount that prevents the occurrence or one or more symptoms of the condition or disorder or is an amount that reduces the severity of, or the length of time during which a subject suffers from, one or more symptoms of the condition or disorder (e.g., by at least 10%, 20%, or 30%, more preferably by at least 50%, 60%, or 70%, and most preferably by at least 80%, 90%, 95%, 99%, or more, relative to a control subject that is not treated with a composition of the disclosure).

Moreover, one skilled in the art will ascertain, a therapeutically effective amount may also refer to the amount of a pharmaceutical composition containing at least one of the phenolic compounds disclosed herein administered to affect COX-1, COX-2, MAO-A, MAO-B, LSD-1 activity of a subject (e.g., by at least 50%, 60%, or 70%, and most preferably by at least 80%, 90%, 95%, 99%, or 100%).

A therapeutically effective amount or effective amount of the compositions containing at least one of the phenolic compounds disclosed herein, used to practice the methods described herein (e.g., the treatment or prophylaxis of disorders or conditions that can be affected by COX-1, COX-2, MAO-A, MAO-B, LSD-1 activities), varies depending upon the nature of the particular disorder or condition that can be determined by standard clinical techniques, route of administration and the age, body weight, and general health of the subject being treated. Ultimately, the prescribers or researchers will decide the appropriate amount and dosage. In some aspects, in vitro assays are optionally employed to help identify optimal dosage ranges. The precise dose to be employed should be decided according to the judgment of the practitioner and each subject's circumstances. However, suitable dosage ranges for intravenous administration are generally about 20 to 1000 micrograms of active compound per kilogram body weight. Suitable dosage ranges for intranasal administration are generally about 0.01 pg/kg body weight to 1 mg/kg body weight. Effective doses may be extrapolated from dose response curves derived from in vitro or animal model test systems. Suppositories generally contain the active ingredient(s) in the range of 0.5% to 10% by weight; oral formulations preferably contain 10% to 95% active ingredient.

By “pharmaceutical composition” the phenolic compounds disclosed herein provide the therapeutically or biologically active agent for formulation into a suitable delivery means for administration to a subject. For the purposes of this disclosure, pharmaceutical or food additive compositions suitable for delivering the phenolic compounds can include, but not limited to, tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of the formulations can be prepared by well-known and accepted methods of art.

Various uses of oleocanthal analogs are described in U.S. Pat. No. 8,586,632 and US Patent Publication 2009/0076142 A1. Since the phenolic compounds disclosed herein are found to be similar to oleocanthal, oleuropein, or their analogs, the use of the phenolic compounds disclosed here are expected to be used in a similar manner and for similar purposes. U.S. Pat. No. 8,586,632 and US Patent Publication 2009/0076142 A1 are herein incorporated by reference.

In one aspect, provided herein is a method of inhibiting COX-1, COX-2, MAO, and/or LSD-1 activity comprising administering an effective amount of a composition, wherein the composition comprises a compound having any one of the following formulas I-VI or their stereo-isomers:

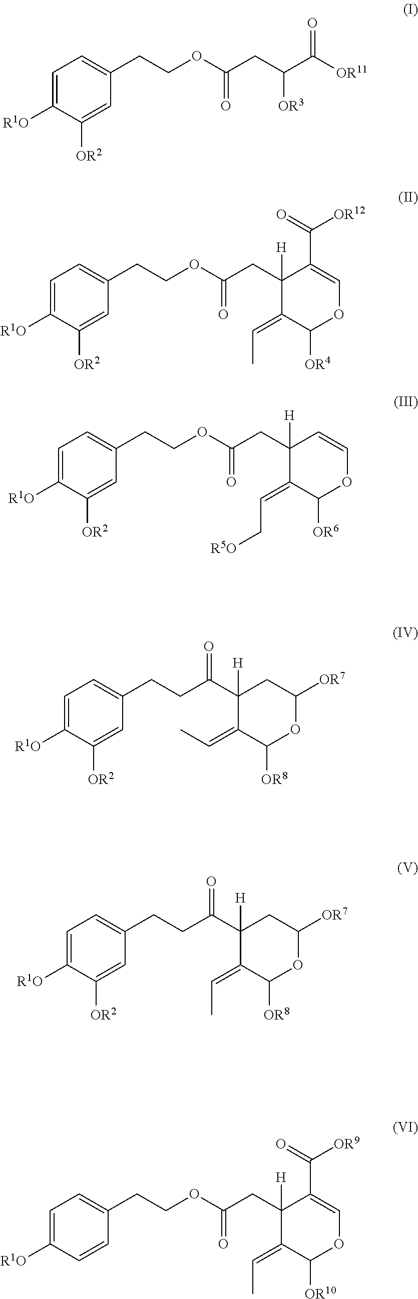

wherein R¹-R¹² are independently H or R²⁰; and R²⁰ is a H, C₁-C₅ alkyl, or a glycoside.

As used herein, a “stereo-isomer” refers to any structures that are different from those drawn as a result of the orientation differences at one or more chiral centers, cis/trans configuration of one or more double-bonds, positional differences of —OR¹ or —OR² groups in the aromatic rings, or other similar differences in R¹-R¹² groups.

In some embodiments, the composition further comprises a pharmaceutically acceptable carrier.

In some embodiments, the compound disclosed herein is one having any one of the following formulas Ia-VIa, stereo-isomer thereof, or salt thereof.

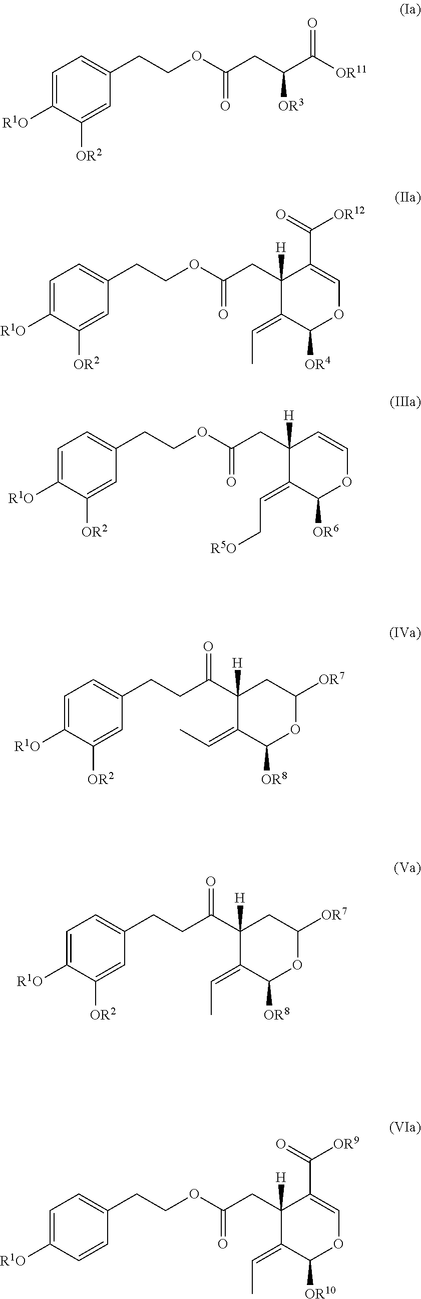

In some other embodiments, the compound disclosed herein is one having any one of the following formulas Ib-VIb, stereo-isomer thereof, or salt thereof.

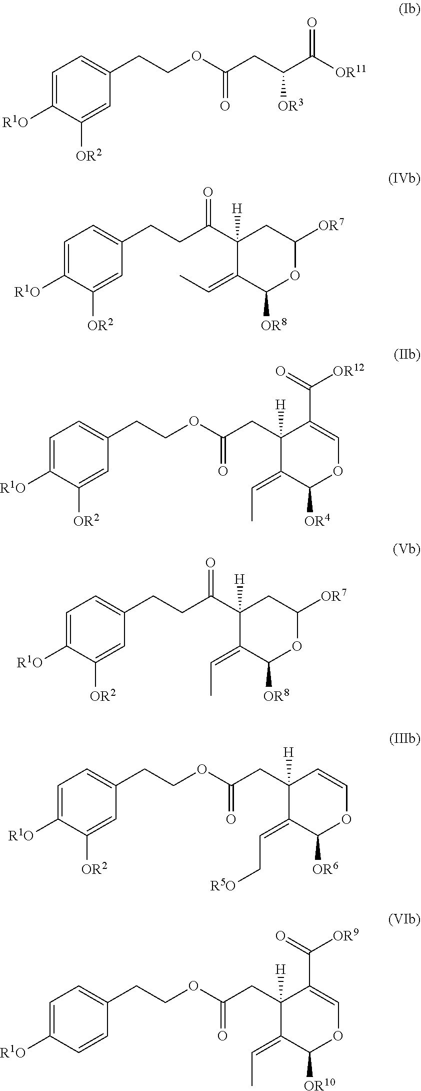

In yet some other embodiments, the compound disclosed herein is one having any one of the following formulas IIc-VIc, stereo-isomer thereof, or salt thereof.

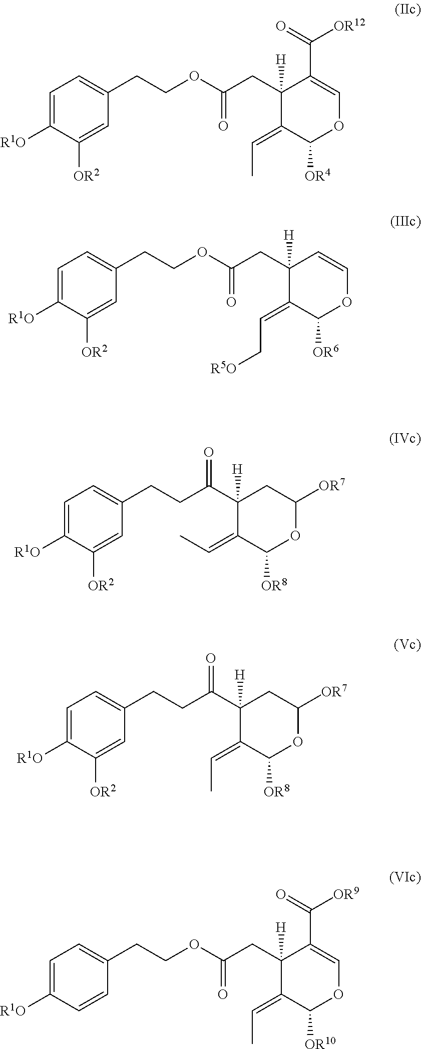

In some embodiments, the compound disclosed herein is one having any one of the following formulas IId-VId, stereo-isomer thereof, or salt thereof.

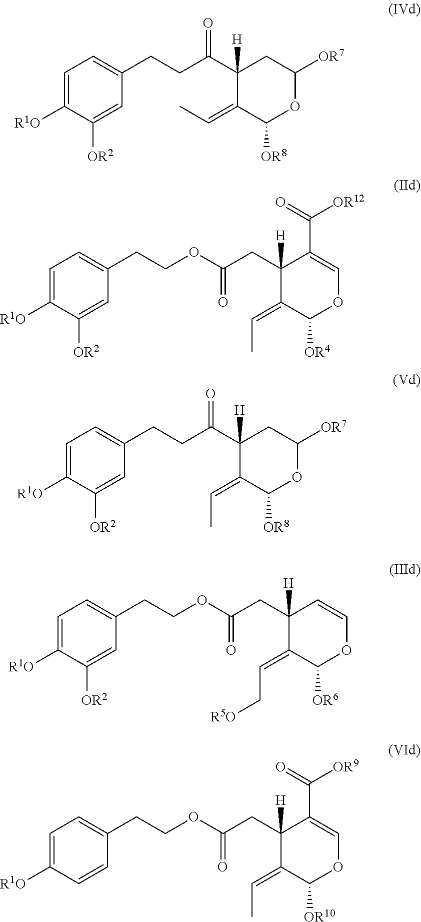

In some embodiments, the compound disclosed here is one having any one of the formulas Ia-VIa, Ib-VIb, IIc-VIc, and IId-VId, stereo-isomer thereof, or salt thereof, wherein only one of R¹-R¹² in the molecule is a C₁-C₅ alkyl or a glycoside and the rest of R¹-R¹² are H. In yet some embodiments, the compound disclosed here is one having any one of the formulas Ia-VIa, Ib-VIb, IIc-VIc, and IId-VId, stereo-isomer thereof, or salt thereof, wherein two of R¹-R¹² in the molecule is are C₁-C₅ alkyl or a glycoside and the rest of R¹-R¹² are H. In some other embodiments, the compound disclosed here is one having any one of the formulas Ia-VIa, Ib-VIb, IIc-VIc, and IId-VId, stereo-isomer thereof, or salt thereof, wherein three of R¹-R¹² in the molecule are C₁-C₅ alkyl or a glycoside and the rest of R¹-R¹² are H. In yet some other embodiments, the compound disclosed here is one having any one of the formulas Ia-VIa, Ib-VIb, IIc-VIc, and IId-VId, stereo-isomer thereof, or salt thereof, wherein four of R¹-R¹² in the molecule are C₁-C₅ alkyl or a glycoside and the rest of R¹-R¹² are H.

In some embodiments, the compound disclosed here is one having any one of the formulas Ia-VIa, Ib-VIb, IIc-VIc, and IId-VId, stereo-isomer thereof, or salt thereof, wherein all R¹-R¹² in the molecule is H, a C₁-C₅ alkyl, methyl, —CH₂CH₃, —CH(CH₃)₂, —C(CH₃)₃, —C(OH)(CH₃)₂, or a glycoside.

In some embodiments, the compound disclosed here is one having any one of the formulas Ia-VIa, Ib-VIb, IIc-VIc, and IId-VId, stereo-isomer thereof, or salt thereof, wherein only one of R¹-R¹² in the molecule is methyl and the rest of R¹-R¹² are H. In yet some embodiments, the compound disclosed here is one having any one of the formulas Ia-VIa, Ib-VIb, IIc-VIc, and IId-VId, stereo-isomer thereof, or salt thereof, wherein two of R¹-R¹² in the molecule are methyl and the rest of R¹-R¹² are H. In some other embodiments, the compound disclosed here is one having any one of the formulas Ia-VIa, Ib-VIb, IIc-VIc, and IId-VId, stereo-isomer thereof, or salt thereof, wherein three of R¹-R¹² in the molecule are methyl and the rest of R¹-R¹² are H. In yet some other embodiments, the compound disclosed here is one having any one of the formulas Ia-VIa, Ib-VIb, IIc-VIc, and IId-VId, stereo-isomer thereof, or salt thereof, wherein four of R¹-R¹² in the molecule are methyl and the rest of R¹-R¹² are H.

In some embodiments, the compound disclosed here is one having any one of the formulas Ia-VIa, Ib-VIb, IIc-VIc, and IId-VId, stereo-isomer thereof, or salt thereof, wherein one of R¹-R¹² in the molecule is a glycoside and the rest of R¹-R¹² are H or C₁-C₅ alkyl. In yet some embodiments, the compound disclosed here is one having any one of the formulas Ia-VIa, Ib-VIb, IIc-VIc, and IId-VId, stereo-isomer thereof, or salt thereof, wherein two of R¹-R¹² in the molecule are glycoside and the rest of R¹-R¹² are H or C₁-C₅ alkyl. In some other embodiments, the compound disclosed here is one having any one of the formulas Ia-VIa, Ib-VIb, IIc-VIc, and IId-VId, stereo-isomer thereof, or salt thereof, wherein three of R¹-R¹² in the molecule are glycoside and the rest of R¹-R¹² are H or C₁-C₅ alkyl. In yet some other embodiments, the compound disclosed here is one having any one of the formulas Ia-VIa, Ib-VIb, IIc-VIc, and IId-VId, stereo-isomer thereof, or salt thereof, wherein four of R¹-R¹² in the molecule are glycosides and the rest of R¹-R¹² are H or C₁-C₅ alkyl.

In some other embodiments, the compound disclosed herein is one having any one of the following formulas Ic, IIe, IIf, IIIe, and IIIf, stereo-isomer thereof, or salt thereof.

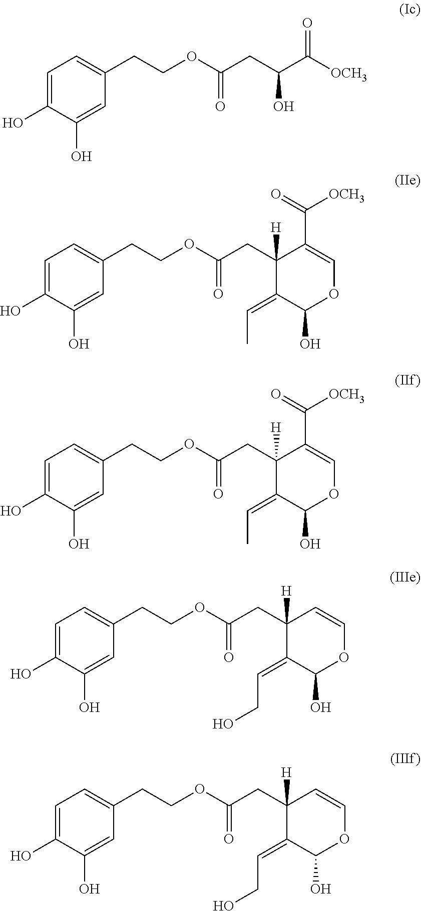 [SMILES: COC(=O)[C@@H](O)CC(=O)OCCc1ccc(O)c(O)c1 ; C/C=C1\[C@H](O)OC=C(C(=O)OC)[C@H]1CC(=O)OCCc1ccc(O)c(O)c1 ; C/C=C1\[C@H](O)OC=C(C(=O)OC)[C@@H]1CC(=O)OCCc1ccc(O)c(O)c1 ; O=C(C[C@@H]1C=CO[C@@H](O)/C1=C\CO)OCCc1ccc(O)c(O)c1 ; O=C(C[C@@H]1C=CO[C@H](O)/C1=C\CO)OCCc1ccc(O)c(O)c1]

In yet some other embodiments, the compound disclosed herein is one having any one of the following formula Id, IIg, IIh, IIIg, and IIIh, stereo-isomer thereof, or salt thereof.

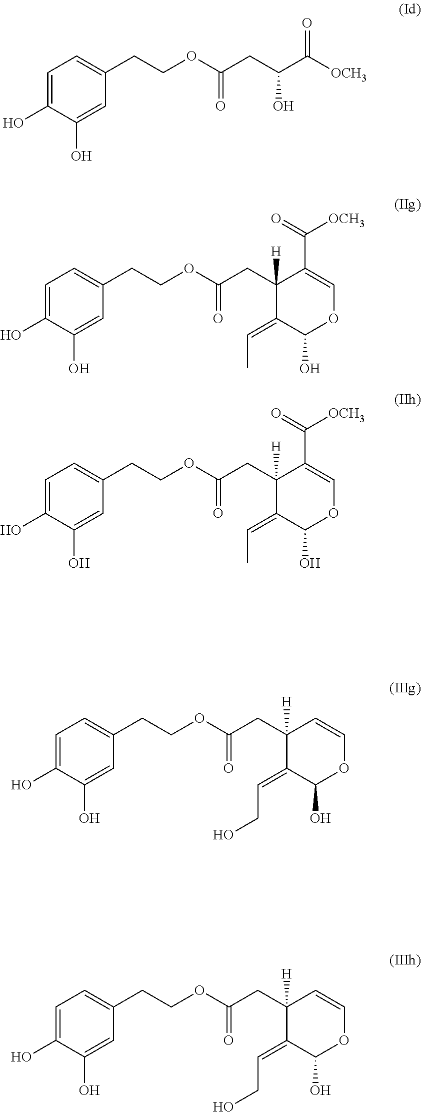 [SMILES: COC(=O)[C@H](O)CC(=O)OCCc1ccc(O)c(O)c1 ; C/C=C1/[C@H](CC(=O)OCCc2ccc(O)c(O)c2)C(C(=O)OC)=CO[C@@H]1O ; C/C=C1\[C@@H](O)OC=C(C(=O)OC)[C@@H]1CC(=O)OCCc1ccc(O)c(O)c1 ; O=C(C[C@H]1C=CO[C@@H](O)/C1=C\CO)OCCc1ccc(O)c(O)c1 ; O=C(C[C@H]1C=CO[C@H](O)/C1=C\CO)OCCc1ccc(O)c(O)c1]

In some embodiments, the compound disclosed herein is one having any one of the following formulas, stereo-isomer thereof, or salt thereof.

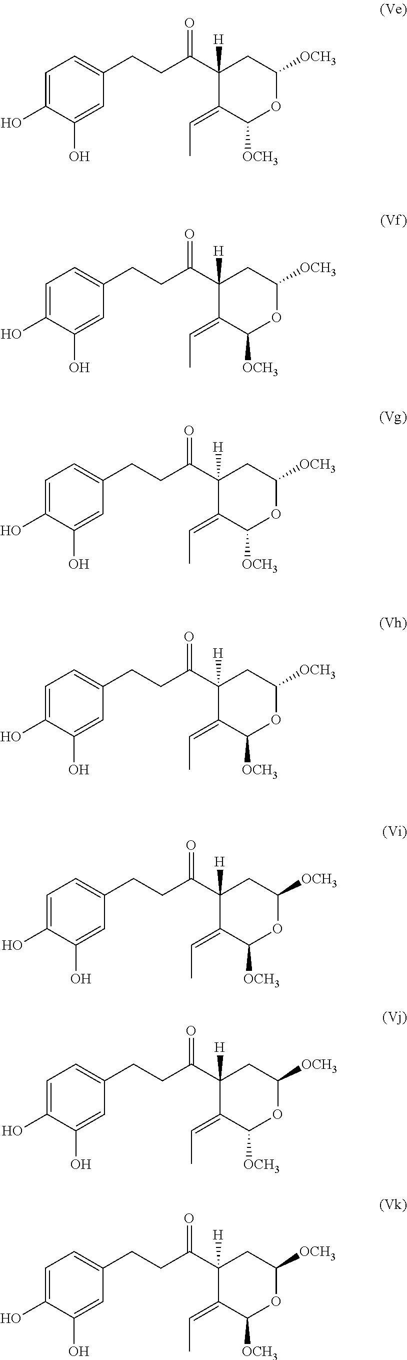

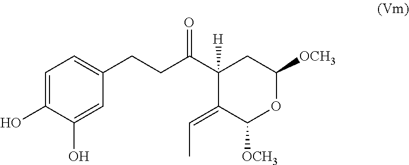 [SMILES: C/C=C1\[C@@H](OC)O[C@H](OC)C[C@@H]1C(=O)CCc1ccc(O)c(O)c1]

In some embodiments, the compound disclosed herein is one having any one of the following formulas, stereo-isomer thereof, or salt thereof.

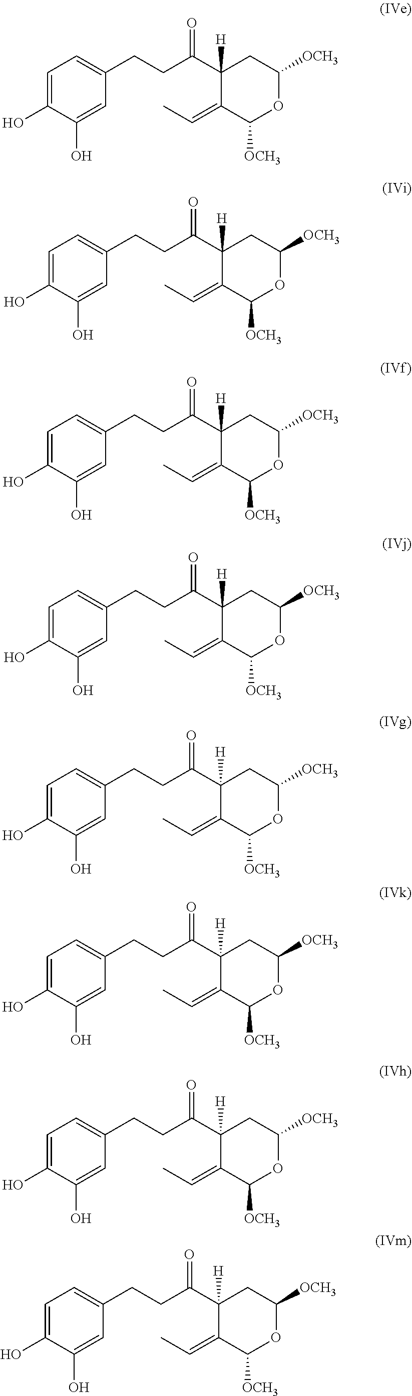

In some embodiments, the compound disclosed herein is one having any one of the following formulas, stereo-isomer thereof, or salt thereof.

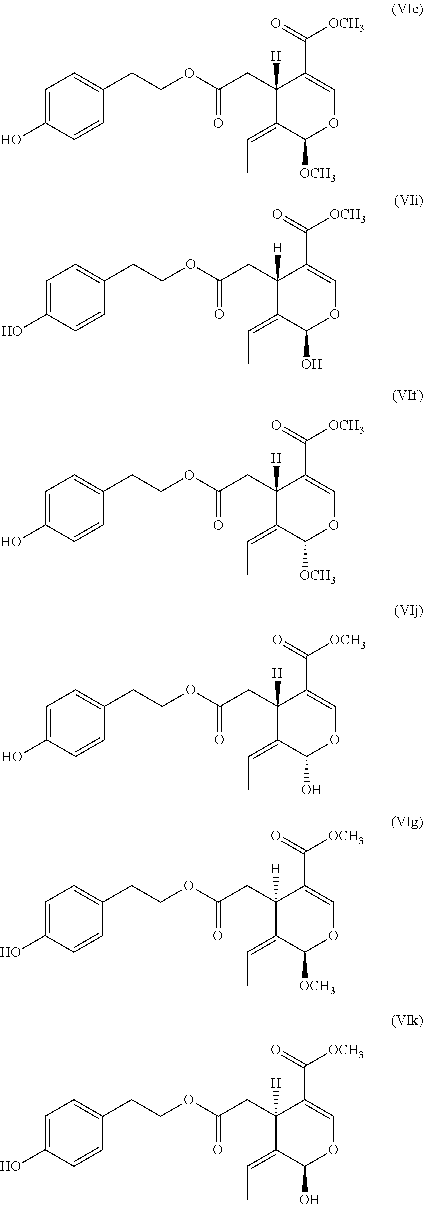 [SMILES: C/C=C1\[C@H](OC)OC=C(C(=O)OC)[C@H]1CC(=O)OCCc1ccc(O)cc1 ; C/C=C1\[C@H](O)OC=C(C(=O)OC)[C@H]1CC(=O)OCCc1ccc(O)cc1 ; C/C=C1\[C@@H](OC)OC=C(C(=O)OC)[C@H]1CC(=O)OCCc1ccc(O)cc1 ; C/C=C1/[C@H](CC(=O)OCCc2ccc(O)cc2)C(C(=O)OC)=CO[C@@H]1O ; C/C=C1\[C@H](OC)OC=C(C(=O)OC)[C@@H]1CC(=O)OCCc1ccc(O)cc1 ; C/C=C1\[C@H](O)OC=C(C(=O)OC)[C@@H]1CC(=O)OCCc1ccc(O)cc1]

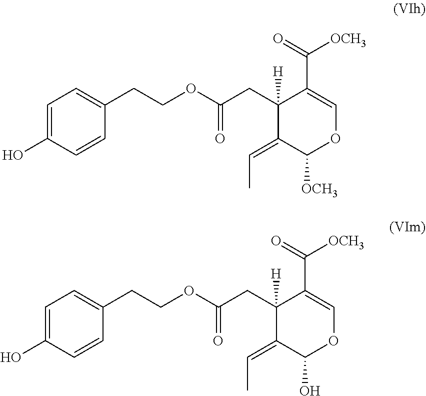 [SMILES: C/C=C1\[C@@H](OC)OC=C(C(=O)OC)[C@@H]1CC(=O)OCCc1ccc(O)cc1 ; C/C=C1\[C@@H](O)OC=C(C(=O)OC)[C@@H]1CC(=O)OCCc1ccc(O)cc1]

In some embodiments, the compound disclosed herein is one having any one of the formulas I, Ia-Ic, stereo-isomer thereof, or salt thereof.

In some embodiment, the compound disclosed herein is

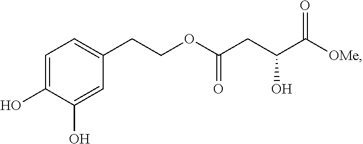 [SMILES: COC(=O)[C@H](O)CC(=O)OCCc1ccc(O)c(O)c1]

methyl malate beta-hydroxytyrosol ester.

In some embodiments, the compound inhibits COX-1 activity in the patent. In some other embodiments, the compound inhibits COX-2 activity. In yet some other embodiments, the compound inhibits COX-1 and COX-2 activity in the patent. In some embodiments, the compound inhibits MAO activity in the patent. In some other embodiments, the compound inhibits MAO-A activity in the patent. In yet some other embodiments, the compound inhibits MAO-B activity in the patent. In some embodiments, the compound inhibits MAO-A and MAO-B activity in the patent. In some other embodiments, the compound inhibits LSD-1 activity in the patent. In some other embodiments, the compound inhibits COX-1, COX-2, MAO and LSD-1 activity in the patent.

In another aspect, provided herein is a composition comprising a therapeutically effective amount of any one or more of the compounds disclosed above or salts thereof and a pharmaceutically acceptable carrier.

In some embodiments, the composition comprises a therapeutically effective amount of a compound having any of the formula I-VI, stereo-isomer thereof, or salt thereof and a pharmaceutically acceptable carrier.

In some embodiments, the compound disclosed herein is one having any one of the formulas Ia-VIa, stereo-isomer thereof, or salt thereof.

In some other embodiments, the compound disclosed herein is one having any one of the formulas Ib-VIb, stereo-isomer thereof, or salt thereof.

In yet some other embodiments, the compound disclosed herein is one having any one of the formulas Ic-VIc, stereo-isomer thereof, or salt thereof.

In some embodiments, the compound disclosed herein is one having any one of the formulas IId-VId, stereo-isomer thereof, or salt thereof.

In some other embodiments, the compound disclosed herein is one having any one of the formulas Ic, IIe, IIf, IIIe, and, IIIf, stereo-isomer thereof, or salt thereof.

In yet some other embodiments, the compound disclosed herein is one having any one of the formulas Id, IIg, IIh, IIIg, and, IIIh, stereo-isomer thereof, or salt thereof.

In some embodiments, the compound disclosed herein is one having any one of the formulas Ve-Vm, stereo-isomer thereof, or salt thereof.

In some embodiments, the compound disclosed herein is one having any one of the formulas IVe-IVm, stereo-isomer thereof, or salt thereof.

In some embodiments, the compound disclosed herein is one having any one of the formulas VIe-VIm, stereo-isomer thereof, or salt thereof.

In some embodiments, the compound disclosed herein is one having any one of the formulas I, Ia-Ic, stereo-isomer thereof, or salt thereof.

In some embodiments, the compound is methyl malate beta-hydroxytyrosol ester,

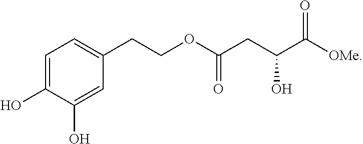 [SMILES: COC(=O)[C@H](O)CC(=O)OCCc1ccc(O)c(O)c1]

In some embodiments, the composition inhibits COX-1 activity in the patent. In some other embodiments, the compound inhibits COX-2 activity. In yet some other embodiments, the composition inhibits COX-1 and COX-2 activity in the patent. In some embodiments, the composition inhibits MAO activity in the patent. In some other embodiments, the composition inhibits MAO-A activity in the patent. In yet some other embodiments, the composition inhibits MAO-B activity in the patent. In some embodiments, the composition inhibits MAO-A and MAO-B activity in the patent. In some other embodiments, the composition inhibits LSD-1 activity in the patent. In some other embodiments, the composition inhibits COX-1, COX-2, MAO and LSD-1 activity in the patent.

In another aspect, provided herein is method of use comprising administering to a subject a composition comprising one or more compounds disclosed herein and above or their salts and a pharmaceutically acceptable carrier.

In some embodiments, the composition inhibits COX-1, COX-2, MAO-A, MAO-B, LSD-1 activity, or a combination thereof.

In yet another aspect, provided here is a method of inhibiting MAO activity in a patient, wherein the method comprises administering to a patient an effective amount of a composition having one or more compounds disclosed herein and above or their salts and a pharmaceutically acceptable carrier.

In some embodiments, the composition inhibits MAO-A activity. In some other embodiments, the composition inhibits MAO-B activity. In yet some other embodiments, the composition inhibits MAO-A and MAO-B activity.

In one aspect, provided here is a method of inhibiting LSD-1 activity in a patient, wherein the method comprises administering an effective amount of a composition having one or more compounds disclosed herein and above or their salts and a pharmaceutically acceptable carrier.

In yet another aspect, provided herein is a method of treating a patient with an inflammatory disorder comprising administering to a patient with an inflammatory disorder an effective amount of a composition comprising one or more compounds disclosed herein and above or their salts and a pharmaceutically acceptable carrier.

In one aspect, provided herein is a method of treating a patient with a neurodegenerative disorder, wherein the method comprises administering to a patient with a neurodegenerative disorder an effective amount of a composition comprising one or more compounds disclosed herein and above or their salts and a pharmaceutically acceptable carrier.

In yet another aspect, provided herein is a method of treating a patient with a depression disorder, wherein the method comprises administering to a patient with a depression disorder an effective amount of a composition comprising a compound having one or more compounds disclosed herein and above or their salts and a pharmaceutically acceptable carrier.

In some embodiments, the method further comprises co-administering an effective amount of an antidepressant and/or other depression treatments including, for examples, tricyclic, 3,4-Methylenedioxymethamphetamine (MDMA, or ecstasy), meperidine, tramadol, dextromethorphan, or combination thereof.

In yet another aspect, provided herein is a method of treating a patient with cancer, wherein the method comprises administering to a patient with cancer an effective amount of a composition comprising a compound having one or more compounds disclosed herein and above or their salts and a pharmaceutically acceptable carrier.

In some embodiments, the composition comprises a compound having any of the formulas I-VI, stereo-isomer thereof, or salt thereof and a pharmaceutically acceptable carrier.

In some embodiments, the compound is one having any one of the formulas Ia-VIa, stereo-isomer thereof, or salt thereof.

In some other embodiments, the compound is one having any one of the formulas Ib-VIb, stereo-isomer thereof, or salt thereof.

In yet some other embodiments, the compound is one having any one of the formulas Ic-VIc, stereo-isomer thereof, or salt thereof.

In some embodiments, the compound is one having any one of the formulas IId-VId, stereo-isomer thereof, or salt thereof.

In some embodiments, the compound is one having any one of the formulas Ia-VIa, Ib-VIb, IIc-VIc, and IId-VId, stereo-isomer thereof, or salt thereof, wherein only one of R¹-R¹² in the molecule is a C₁-C₅ alkyl or a glycoside and the rest of R¹-R¹² are H. In yet some embodiments, the compound is one having any one of the formulas Ia-VIa, Ib-VIb, IIc-VIc, and IId-VId, stereo-isomer thereof, or salt thereof, wherein two of R¹-R¹² in the molecule is are C₁-C₅ alkyl or a glycoside and the rest of R¹-R¹² are H. In some other embodiments, the compound is one having any one of the formulas Ia-VIa, Ib-VIb, IIc-VIc, and IId-VId, stereo-isomer thereof, or salt thereof, wherein three of R¹-R¹² in the molecule are C₁-C₅ alkyl or a glycoside and the rest of R¹-R¹² are H. In yet some other embodiments, the compound is one having any one of the formulas Ia-VIa, Ib-VIb, IIc-VIc, and IId-VId, stereo-isomer thereof, or salt thereof, wherein four of R¹-R¹² in the molecule are C₁-C₅ alkyl or a glycoside and the rest of R¹-R¹² are H.

In some embodiments, the compound is one having any one of the formulas Ia-VIa, Ib-VIb, IIc-VIc, and IId-VId, stereo-isomer thereof, or salt thereof, wherein all R¹-R¹² in the molecule is H, a C₁-C₅ alkyl, methyl, —CH₂CH₃, —CH(CH₃)₂, —C(CH₃)₃, —C(OH)(CH₃)₂, or a glycoside.

In some embodiments, the compound is one having any one of the formulas Ia-VIa, Ib-VIb, IIc-VIc, and IId-VId, stereo-isomer thereof, or salt thereof, wherein only one of R¹-R¹² in the molecule is methyl and the rest of R¹-R¹² are H. In yet some embodiments, the compound is one having any one of the formulas Ia-VIa, Ib-VIb, IIc-VIc, and IId-VId, stereo-isomer thereof, or salt thereof, wherein two of R¹-R¹² in the molecule are methyl and the rest of R¹-R¹² are H. In some other embodiments, the compound is one having any one of the formulas Ia-VIa, Ib-VIb, IIc-VIc, and IId-VId, stereo-isomer thereof, or salt thereof, wherein three of R¹-R¹² in the molecule are methyl and the rest of R¹-R¹² are H. In yet some other embodiments, the compound is one having any one of the formulas Ia-VIa, Ib-VIb, IIc-VIc, and IId-VId, stereo-isomer thereof, or salt thereof, wherein four of R¹-R¹² in the molecule are methyl and the rest of R¹-R¹² are H.

In some embodiments, the compound is one having any one of the formulas Ia-VIa, Ib-VIb, IIc-VIc, and IId-VId, stereo-isomer thereof, or salt thereof, wherein one of R¹-R¹² in the molecule is a glycoside and the rest of R¹-R¹² are H or C₁-C₅ alkyl. In yet some embodiments, the compound is one having any one of the formulas Ia-VIa, Ib-VIb, IIc-VIc, and IId-VId, stereo-isomer thereof, or salt thereof, wherein two of R¹-R¹² in the molecule are glycoside and the rest of R¹-R¹² are H or C₁-C₅ alkyl. In some other embodiments, the compound is one having any one of the formulas Ia-VIa, Ib-VIb, IIc-VIc, and IId-VId, stereo-isomer thereof, or salt thereof, wherein three of R¹-R¹² in the molecule are glycoside and the rest of R¹-R¹² are H or C₁-C₅ alkyl. In yet some other embodiments, the compound is one having any one of the formulas Ia-VIa, Ib-VIb, IIc-VIc, and IId-VId, stereo-isomer thereof, or salt thereof, wherein four of R¹-R¹² in the molecule are glycosides and the rest of R¹-R¹² are H or C₁-C₅ alkyl.

In some other embodiments, the compound is one having any one of the formulas Ic, IIe, IIf, IIIe, and, IIIf, stereo-isomer thereof, or salt thereof.

In yet some other embodiments, the compound is one having any one of the formula Id, IIg, IIh, IIIg, and, IIIh, stereo-isomer thereof, or salt thereof.

In some embodiments, the compound is one having any one of the formulas Ve-Vm, stereo-isomer thereof, or salt thereof.

In some embodiments, the compound is one having any one of the formulas IVe-IVm, stereo-isomer thereof, or salt thereof.

In some embodiments, the compound is one having any one of the formulas VIe-VIm, stereo-isomer thereof, or salt thereof.

In some embodiments, the compound is one having any one of the formulas I, Ia-Ic, stereo-isomer thereof, or salt thereof.

In some embodiments, the compound is methyl malate beta-hydroxytyrosol ester,

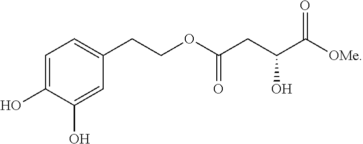 [SMILES: COC(=O)[C@H](O)CC(=O)OCCc1ccc(O)c(O)c1]

In some embodiments, the inflammatory disorder is psoriasis, cancer, asthma, allergic rhinitis, respiratory distress syndrome, inflammatory bowel disease, Chron's disease, gastritis, irritable bowel syndrome, ulcerative colitis, migraine, periarteritis nodosa, thyroiditis, aplastic anemia, Hodgkin's disease, sclerodoma, type I diabetes, myasthenia gravis, multiple sclerosis, sorcoidosis, ischemic kidney disease, nephrotic syndrome, Bechet's syndrome, polymyositis, gingivitis, conjunctivitis, vascular disease myocardial ischemia, heart disease, stroke, or combinations thereof.

In yet another aspect, provided here is an antioxidant composition comprising a therapeutically effective amount of one or more of the compounds disclosed herein or their salts, a compound having any of the formulas I-VI, Ia-VIa, Ib-VIb, IIc-VIc, IId-VId, Ic, He, IIf, IIIe, IIIf; Id, IIg, IIh, IIIg, IIIh, Ve-Vm, IVe-IVm, or VIe-VIm, stereo-isomers thereof, or salts thereof and a pharmaceutically acceptable carrier.

In another aspect, provided here is a method of enhancing the flavor of food comprising adding to food an effective amount of one or more of the compounds disclosed herein, a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof, or salts thereof.

In another further aspect, provided here is an animal repellent composition comprising an effective amount of a compound of one or more of the compounds disclosed herein, a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof, or salts thereof and a pharmaceutically acceptable carrier.

In another aspect, provided here is a method for treating a sore throat comprising administering to a patient with a sore throat an effective amount of a composition comprising a pharmaceutically acceptable carrier, or a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof disclosed herein, or salts thereof and a pharmaceutically acceptable carrier.

In yet another aspect, provided herein is a method of preserving food comprising contacting a food with a composition, wherein the composition comprises an effective amount of one or more of the compounds disclosed herein, or a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof, or salts thereof.

In another aspect, provided herein is a method of repelling animals from an edible source comprising adding to an edible source an effective amount of one or more of the compounds disclosed herein, a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof, or salts thereof.

In yet another aspect, provided herein is a method of inhibiting sweetness perception in an edible source comprising adding to an edible source a sweetness inhibiting amount of one or more of the compounds disclosed herein, or a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof, or salts thereof.

In another aspect, provided herein is a method of treating a cold comprising administering to a patient in need of treatment a composition comprising an effective amount of one or more of the compounds disclosed herein, or a compound having any of the formula I-VI, Ia-VIa, Ib-VIb, IIc-VIc, IId-VId, Ic, IIe, IIf, IIIe, IIIf; Id, IIg, IIh, IIIg, IIIh, Ve-Vm, IVe-IVm, or VIe-VIm, stereo-isomers thereof, or salts thereof.

In yet another aspect, provided herein is a method of inhibiting growth of microorganisms comprising contacting microorganisms with an effective amount of a composition comprising one or more of the compounds disclosed herein, or a compound having any of the formulas I-VI, Ia-VIa, Ib-VIb, IIc-VIc, IId-VId, Ic, IIe, IIf, IIIe, IIIf; Id, IIg, IIh, IIIg, IIIh, Ve-Vm, IVe-IVm, or VIe-VIm, stereo-isomers thereof, or salts thereof.

In another aspect, provided herein is a method of treating pain in a patient comprising administering to a patient an effective amount of a composition comprising one or more of the compounds disclosed herein, or a compound having any of the formulas I-VI, Ia-VIa, Ib-VIb, IIc-VIc, IId-VId, Ic, IIe, IIf, IIIe, IIIf, Id, IIg, IIh, IIIg, IIIh, Ve-Vm, IVe-IVm, or VIe-VIm, stereo-isomers thereof, or salts thereof.

In yet another aspect, provided herein is a method of preventing a neurodegenerative disorder comprising administering to a patient an effective amount of a composition comprising one or more of the compounds disclosed herein, or a compound having any of the formulas I-VI, Ia-VIa, Ib-VIb, IIc-VIc, IId-VId, Ic, IIe, IIf, IIIe, IIIf; Id, IIg, IIh, IIIg, IIIh, Ve-Vm, IVe-IVm, or VIe-VIm, stereo-isomers thereof, or salts thereof.

In some embodiments, the neurodegenerative disorder is Alzheimer's disease, cognitive impairment, or combination thereof.

In some embodiments, the compound disclosed herein is one having any one of the formulas I, Ia-Ic, stereo-isomer thereof, or salt thereof.

For the methods and compositions disclosed herein, in some embodiments, the method or composition inhibits COX-1, COX-2, MAO-A, MAO-B, LSD-1 activity, or a combination thereof in the patient or subject. In some embodiments, the composition or method inhibits COX-1 activity in the patent. In some other embodiments, the compound or method inhibits COX-2 activity. In yet some other embodiments, the composition or method inhibits COX-1 and COX-2 activity in the patent. In some embodiments, the composition or method inhibits MAO activity in the patent. In some other embodiments, the composition or method inhibits MAO-A activity in the patent. In yet some other embodiments, the composition or method inhibits MAO-B activity in the patent. In some embodiments, the composition or method inhibits MAO-A and MAO-B activity in the patent. In some other embodiments, the composition or method inhibits LSD-1 activity in the patent. In some other embodiments, the composition or method inhibits COX-1, COX-2, MAO-A, MAO-B, and LSD-1 activity in the patent.

### EXAMPLES

Embodiments of the present disclosure are further defined in the following non-limiting Examples. These Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Screening the Phenolic Compounds in OliveNet Database for Human COX-1 and/or COX-2 Inhibitors

Database for the Phenolic Compounds Derived from Olea Europaea

The database, named OliveNet, describes nearly 700 compounds detected in the olive, including over 200 phenolic compounds from five natural matrices: the olive fruit, leaf, pomace (solid waste), wastewater, and extra virgin oil. Concentration of the compounds and known pharmacological activity of the compounds are included when they become available, with references to biological efficacy determined by in vitro or in vivo studies.

In total, the OliveNet database contains 682 distinct chemical compounds derived from Olea europaea. These compounds are divided into 13 main classes: phenols, fatty acids, aliphatic and aromatic alcohols, sterols, phospholipids, triterpenic acids, volatiles, hydrocarbons, sugars, pigments, tocopherols, amino acids, and other unclassified compounds.

Among the compounds in the database are 223 phenolic compounds derived from Olea Europaea, which are further divided into 13 subclasses: simple phenols, hydroxybenzoic acids, hydroxyphenylacetic acids, hydroxycinnamic acids, flavonoids, lignans, hydroxyisochromans, secoiridoids, coumarins, irridoids, glucosides, methoxyphenols, and phenolic fatty acid esters. The subclasses, numbers, and distributions of the phenolic compounds are listed in Table 1.

TABLE 1
Subclasses of phenolic compounds found in the olive matrix

| Phenol | Total | Matrix composition | | | | |
|---|---|---|---|---|---|---|
| Subclass | number of | Olive | | | Waste- | Extra- |
| Name | compounds | fruit | Leaf | Pomace | water | virgin oil |
| Simple | 19 | 4 | 2 | 7 | 6 | 12 |
| phenols | | | | | | |
| Hydroxy- | 13 | 10 | 2 | 7 | 2 | 8 |
| benzoic acids | | | | | | |
| Hydroxy- | 6 | 4 | — | 2 | 1 | 4 |
| phenylacetic | | | | | | |
| acids | | | | | | |
| Hydroxy- | 14 | 8 | 2 | 11 | 2 | 7 |
| cinnamic acids | | | | | | |
| Flavonoids | 34 | 21 | 15 | 19 | 5 | 6 |
| Lignans | 14 | 4 | 8 | 4 | 2 | 5 |
| Hydroxy- | 2 | — | 2 | — | — | 2 |
| isochromans | | | | | | |
| Secoiridoids | 80 | 44 | 19 | 38 | 8 | 19 |
| Coumarins | 4 | — | 4 | — | — | — |
| Irridoids | 2 | — | | 2 | — | — |
| Glucosides | 29 | 14 | 10 | 22 | 3 | 5 |
| Methoxy- | 4 | — | — | | 3 | 1 |
| phenols | | | | | | |
| Phenolic fatty | 2 | 1 | — | 1 | — | — |
| acid esters | | | | | | |
| Total | 223 | 110 | 64 | 113 | 32 | 69 |
| Phenols | | | | | | |

### Preparation of COX-1 and COX-2 Protein Structures

As there was no x-ray crystal structure available for human COX-1 in Protein Data Bank (PDB), the structure was generated using homology modeling techniques. The amino acid sequence of COX-1 was retrieved from UniProt (UniProt ID: P23219). The template structure was identified using blastp (protein-protein BLAST) algorithm, with the most favorable template structure selected for use (PDB ID:1CQE, 3.1 Å). The homology model of COX-1 was built with MODELLER v9, using the partial sequence (Pro32-Pro583), with ten models generated. The models were evaluated based on the lowest MODELLER zDOPE score and RMSD values. The stereochemical quality of the model was validated using PROCHECK and ProSA. The structure of human COX-2 protein was obtained from PDB, with the accession code 5F1A (2.38 Å).

Prior to docking, the two structures were optimized to adopt energetically stable conformations using Schrodinger's Protein Preparation Wizard. It involves the addition and optimization of hydrogen bonds, termini capping, creation of di-sulfide bonds, followed by restrained minimization using OPLS-AA (2005) forcefield to obtain optimized geometry of the protein.

### Ligand Preparation

The phenolic compounds identified within Olea europaea were prepared for docking studies using the LigPrep utility of Schrodinger's package. It generates all possible tautomeric, stereochemical and ionization variants of the input molecules followed by energy minimization to obtain structures with optimized geometry. The ligands with a molar mass less than 500 g/mol were employed for docking.

### QM-PLD Docking

Ligand-protein docking utilized the quantum mechanics-polarized ligand docking (QM-PLD) protocol from the Schrodinger Maestro Suite. Receptor grid generation of the enzyme active site was conducted within the docking suite of Maestro. The native COX-1 ligand Flurbiprofen (FLP) was centroid to the docking box. The binding site of COX-2 was centered on the active site residues Tyr385, Ser530, Arg120, and Tyr355, since the native bound salicylate was too small to adequately dock larger ligands. An extensive search on the centroid box was performed within 20×20×20 Å of these coordinates.

Docking was carried out using the extra precision (XP) docking protocol of Glide. Default values were utilized for all remaining parameters except where Mulliken charge model was selected to generate QM charges. The most reliable binding pose for each ligand was selected on the basis of calculated van der Waals and electrostatic interactions.

### Binding Mode Analysis

The root-mean-square deviation (RMSD) of the native and docked ligands was measured in Maestro using the superposition feature, whilst the ligand interaction tool was used to analyze ligand-residue contacts. Figures were generated using VMD 1.9.1. All computation works were performed on a Windows 7 workstation equipped with an Intel Core i5 (3.00 GHz) and 8 GB of RAM.

### ADMET Analysis

The absorption, distribution, metabolism, excretion, and toxicity (ADMET) properties of each phenolic compound was measured to assess the pharmacokinetic attributes of the compound within the human body using the ‘ADMET Descriptors’ function within Discovery Studio 4.1 (Biovia Discovery Studio, Velizy-Villacoublay, France). The module is used to quantitatively predict properties by a set of rules that specify ADMET characteristics. For obtained hits, some important ADMET descriptors, for example, human intestinal absorption, aqueous solubility, blood brain barrier, plasma protein binding, and hepatotoxicity were calculated. Human intestinal absorption and aqueous solubility descriptors predict the absorption and solubility, respectively, at different levels. Blood brain barrier, plasma protein binding, and hepatotoxicity descriptors predict blood-brain penetration of a molecule after oral administration, binding to carrier proteins in the blood, and potential human hepatotoxicity. The compounds that fulfilled the acceptable criteria for these descriptors were subsequently selected for molecular docking studies.

### Model Evaluation and Validation

PDB structure 1CQE (ovine ortholog) was selected as the template for the generation of human COX-1, based on having the highest sequence identity (93%) following the BLAST-protein search. The stereochemical quality of the model was examined using Procheck, which demonstrated that 92.6% of the residues were in the most favored regions, whilst 7.4% were within the allowed regions of the Ramachandran plot. This suggested that the quality of the model was comparable to refined structures.

The QMPLD docking protocol was able to reliably predict the position of experimentally observed native ligands, producing an RMSD deviating less than 2 Å from the crystal structures for the native FLP and salicylate ligands in COX-1 and COX-2 proteins, respectively.

### Molecular Docking & Interaction Analysis

159 phenolic compounds with a molecular weight of less than 500 g/mol were identified within Olea europaea and categorized into 13 chemical classes. Among the compounds docked within the cyclooxygenase enzymes, six classes demonstrated the greatest predicted binding affinities overall, comparable to some putative COX inhibitors secoiridoids, hydroxycinnamic acids, flavonoids, glucosides, lignans and phenolic fatty acid esters. Within each compound class, analogous binding modes among the ligands that produced the greatest binding affinities was observed, with specific ligand interactions between prominent residues within the active site of the COX enzyme improving the binding affinity score. Table 2 and Table 3 list the top 20 compounds with greatest Glide Energy, their molecular weights, for COX-1 and COX-2 proteins, respectively, and their subclasses.

TABLE 2
Top 20 greatest Glide Energy for COX-1

| | Glide Energy | | |
|---|---|---|---|
| Ligand | (kcal/mol) | MW | Class |
| 1-oleyltyrosol | −50.865 | 389 | Phenolic fatty acid |
| esters | | | |
| Ligstroside derivative 2 | −48.222 | 453 | Secoiridoids |
| Rosmarinic acid | −47.359 | 361 | Hydroxycinnamic |
| | | | acids |
| Oleuropeindial (keto form) | −45.252 | 378 | Secoiridoids |
| 10-Hydroxy oleuropein | −45.202 | 394 | Secoiridoids |
| aglycone | | | |
| Oleacein | −44.865 | 321 | Secoiridoids |
| Caftaric acid | −44.118 | 312 | Hydroxycinnamic |
| | | | acids |
| demethyloleuropein | −43.261 | 364 | Secoiridoids |
| aglycone (enol form) | | | |
| Deoxyloganic acid lauryl | −43.078 | 408 | Phenolic fatty acid |
| ester | | | esters |
| Methyl malate- | −43.03 | 284 | Secoiridoids |
| hydroxytyrosol ester | | | |
| Apigenin-7-O-glucoside | −42.693 | 432 | Flavonoids |
| p-HPEA-EDA | −42.425 | 304 | Secoiridoids |
| Decarboxymethyl | −42.425 | 304 | Secoiridoids |
| ligstroside aglycone | | | |
| Oleocanthal | −42.425 | 305 | Secoiridoids |
| 3,4-DHPEA-EDA | −42.234 | 320 | Secoiridoids |
| Hydroxytyrosol | −42.071 | 382 | Secoiridoids |
| acyclodihydroelenolate | | | |
| 3,4-dihydroxyphenylethyl | −41.404 | 320 | Hydroxybenzoic |
| 4-formyl-3-formylmethyl-4- | | | acids* |
| Oleuropeindial (enol form) | −41.052 | 378 | Secoiridoids |
| 3,4-DHPEA-DETA | −40.784 | 350 | Secoiridoids |

TABLE 3
Top 20 greatest Glide Energy for COX-2

| | Glide Energy | | |
|---|---|---|---|
| Ligand | (kcal/mol) | MW | Class |
| Ligstroside derivative 2 | −49.682 | 453 | Secoiridoids |
| 1-oleyltyrosol | −43.895 | 389 | Phenolic fatty acid |
| | | | esters |
| Hydroxytyrosol diglucoside | −43.855 | 478 | Glucosides |
| Luteolin-4′-0-glucoside | −42.307 | 448 | Flavonoids |
| 10-Hydroxy-10-methyl | −40.673 | 408 | Secoiridoids |
| oleuropein aglycone | | | |
| Oleuropeindial - Lactone | −40.125 | 378 | Secoiridoids |
| (Cannizzaro-like product of | | | |
| oleuropeindial) | | | |
| Berchemol | −40.041 | 376 | Lignans |
| Hydroxytyrosol-3-β-glucoside | −39.91 | 316 | Glucosides |
| (+)-1-Acetoxypinoresino1-4″- | −39.494 | 431 | Lignans |
| O-methyl ether | | | |
| Oleuropeindial (keto form) | −39.385 | 378 | Secoiridoids |
| Chlorogenic acid | −38.948 | 354 | Hydroxycinnamic |
| | | | acids |
| 10-Hydroxy oleuropein | −38.795 | 394 | Secoiridoids |
| aglycone | | | |
| Luteolin-7-O-glucoside | −38.19 | 448 | Flavonoids |
| Hemiacetal of dialdehydic | −36.487 | 334 | Secoiridoids |
| oleuropein aglycone | | | |
| decarboxymethyl | | | |
| 3,4-DHPEA-EDA | −36.475 | 320 | Secoiridoids |
| Hydroxytyrosol elenolate | −36.375 | 364 | Secoiridoids* |
| Hydroxytyrosil-elenolate | −36.375 | 392 | Secoiridoids |
| Demethyloleuropein aglycone | −36.147 | 364 | Secoiridoids |
| Oleuropeindial (Cannizzaro- | −35.639 | 396 | Secoiridoids |
| like product of oleuropeindial) | | | |

### ADMET Analysis for the Phenolic Compounds

Following docking, the ADMET properties of the olive phenolic compounds were analyzed to determine the bioavailability of the compounds. Out of 222 phenolic compounds, 214 were found to not be inhibitors of cytochrome P450 2D6, 154 to be non-hepatotoxic, and 192 to be low binders of plasma protein. 69 phenolic compounds fell within the 95% confidence limit for intestinal absorption and blood brain barrier penetration.

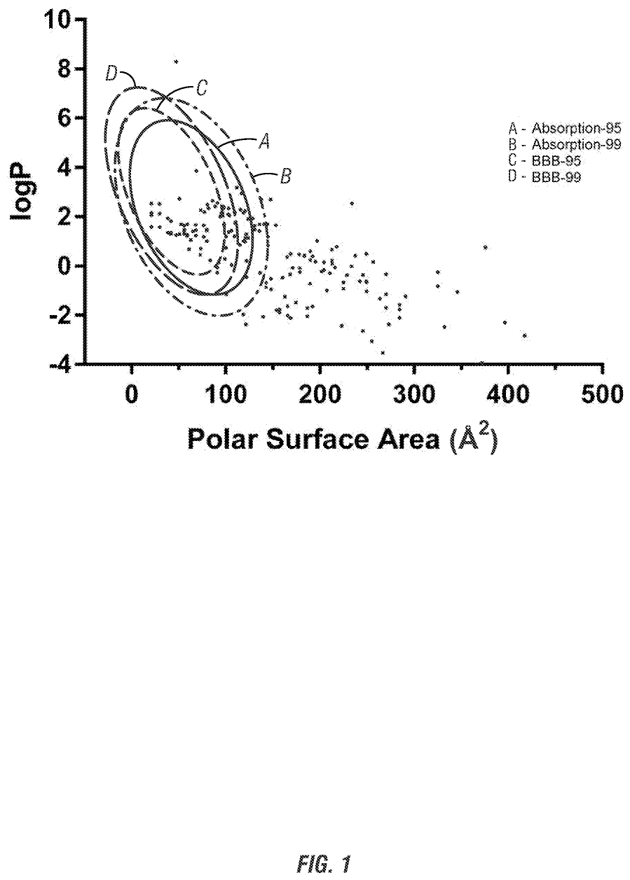

FIG. 1 shows PSA vs log P for the olive phenolics showing 95% and 99% confidence limits, denoted by ellipses corresponding to blood-brain barrier and intestinal absorption models.

### Combining Docking and ADMET Analysis

Combining the results from docking and ADMET analysis, a list of the strong binders that were also ADMET approved was curated and listed in Table 4. The compounds were considered ADMET approved if they fell within both 99 and 95 confidence limits for intestinal absorption. The compounds also penetrating the 99 and 95 confidence limits for blood brain barrier penetration according to Discovery Studio are denoted by \hat{ } and *, respectively in Table 4.

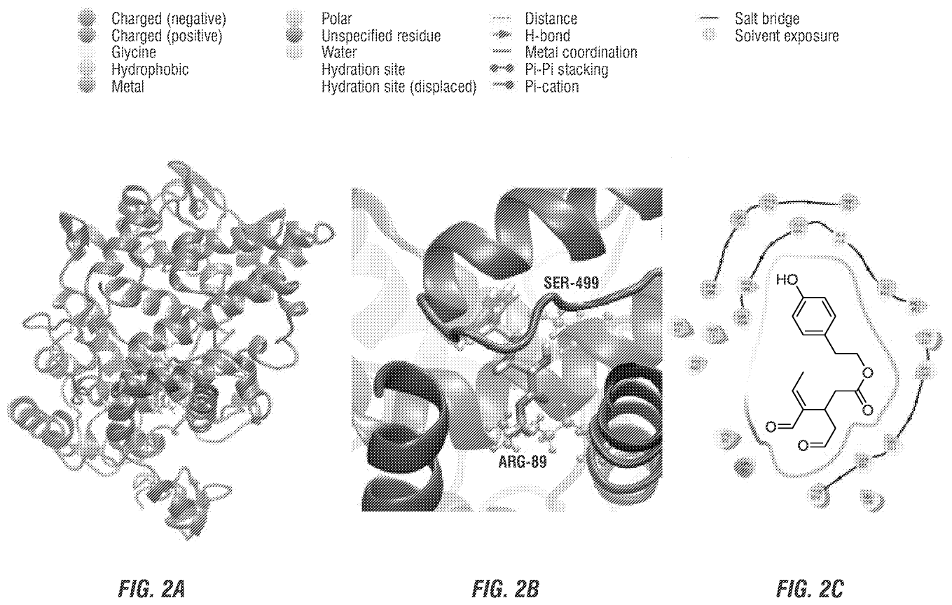

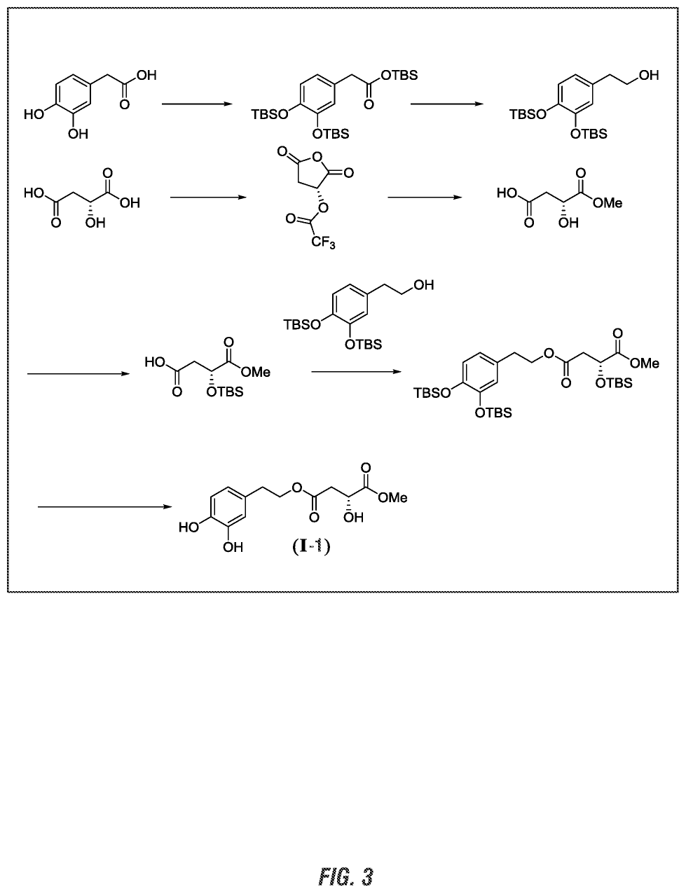

Table 5 lists the top docking, ADMET approved, and non-commercially available compounds, their structures, molecular weights, and COX-2/COX-2 binding rankings, respectively. FIG. 2A-FIG. 2C show oleocanthal docked to COX-1 shown in 3-dimensional and 2-dimensional views. FIG. 2A shows the whole protein with oleocanthal; FIG. 2B highlights oleocanthal in the binding pocket; and FIG. 2C shows a 2D ligand interaction diagram between oleocanthal and residues of the COX-1 protein.

TABLE 4
List of the Top docking compounds that are also ADMET approved

| | COX-1 | COX-1 | COX-2 | COX-2 | |
|---|---|---|---|---|---|
| | Docking | Glide | Docking | Glide | |
| Phenolic ligand | Rank | Energy | Rank | Energy | MW |
| Oleacein (Dialdehydic | 6 | −44.865 | 24 | −34.428 | 321 |
| form of decarboxymethyl | | | | | |
| Oleuropein aglycon) ^{∧} | | | | | |
| Methyl malate- | 10 | −43.03 | 34 | −32.925 | 284 |
| hydroxytyrosol ester | | | | | |
| Decarboxymethyl | 13 | −42.425 | 31 | −33.763 | 304 |
| ligstroside aglycone* | | | | | |
| p-HPEA-EDA* | 12 | −42.425 | 43 | −30.408 | 304 |
| Oleocanthal (Dialdehydic | 14 | −42.425 | 44 | −30.408 | 305 |
| form of decarboxymethyl | | | | | |
| Ligstroside aglycon)* | | | | | |
| 3,4-DHPEA-EDA | 15 | −42.234 | 15 | −36.475 | 320 |
| (Oleuropein-aglycone di- | | | | | |
| aldehyde) ^{∧} | | | | | |
| Ligstroside aglycone | 23 | −40.105 | 20 | −35.28 | 376 |
| methyl acetal * | | | | | |
| Oleuropeindial - Lactone | 35 | −37.689 | 6 | −40.125 | 378 |
| (Cannizzaro-like product | | | | | |
| of oleuropeindial) | | | | | |
| Ligstroside aglycone ^{∧} | 33 | −37.703 | 26 | −34.122 | 380 |
| 10-Hydroxy oleuropein | 22 | −40.335 | 37 | −32.177 | 336 |
| aglycone | | | | | |
| decarboxymethyl | | | | | |
| 3,4-DHPEA-DETA * | 19 | −40.784 | 39 | −31.546 | 350 |
| Oleuropein aglycone | 31 | −38.088 | 32 | −33.702 | 378 |
| (3,4-DHPEA-EA) | | | | | |

TABLE 5
Refined list of top docking ADMET approved and non-commercially available
compounds

| | | COX-1 | COX-2 | |
|---|---|---|---|---|
| | | Docking | Docking | |
| Phenolic ligand | MW | Rank | Rank | Structure |
| Methyl malate- hydroxytyrosol ester | 284 | 10 | 34 | 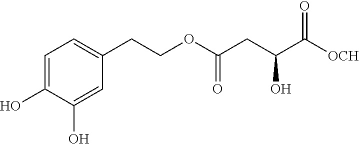 |
| Decarboxymethyl ligstroside aglycone* | 304 | 13 | 31 | 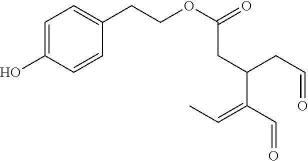 [SMILES: C/C=C(/C=O)C(CC=O)CC(=O)OCCc1ccc(O)cc1] |
| p-HPEA-EDA* | 304 | 12 | 43 | 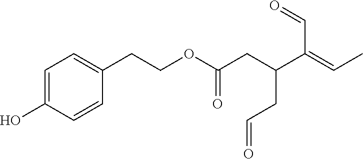 [SMILES: C/C=C(\C=O)C(CC=O)CC(=O)OCCc1ccc(O)cc1] |
| Oleocanthal (Dialdehydic form of decarboxymethyl Ligstroside aglycon)* | 305 | 14 | 44 | 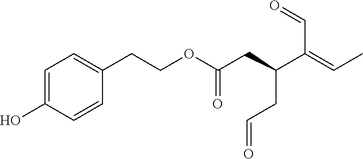 [SMILES: C/C=C(\C=O)[C@@H](CC=O)CC(=O)OCCc1ccc(O)cc1] |
| 3,4-DHPEA- EDA (Oleuropein- aglycone di- aldehyde) \hat{ } | 320 | 15 | 15 | 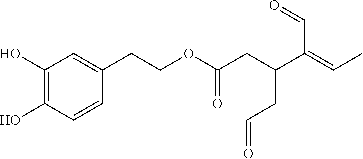 [SMILES: C/C=C(\C=O)C(CC=O)CC(=O)OCCc1ccc(O)c(O)c1] |
| Ligstroside aglycone methyl acetal * | 376 | 23 | 20 | 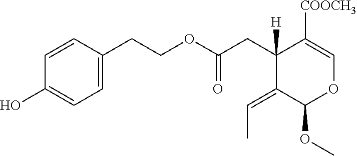 [SMILES: C/C=C1\[C@H](OC)OC=C(C(=O)OC)[C@H]1CC(=O)OCCc1ccc(O)cc1] |
| Oleuropeindial- Lactone (Cannizzaro- like product of oleuropeindial) | 378 | 35 | 6 | 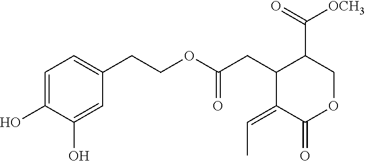 [SMILES: C/C=C1\C(=O)OCC(C(=O)OC)C1CC(=O)OCCc1ccc(O)c(O)c1] |
| Ligstroside aglycone \hat{ } | 380 | 33 | 26 | 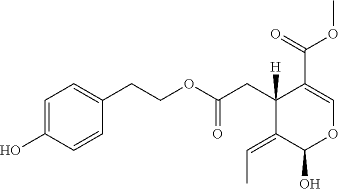 [SMILES: C/C=C1\[C@H](O)OC=C(C(=O)OC)[C@H]1CC(=O)OCCc1ccc(O)cc1] |
| 10-Hydroxy oleuropein aglycone decarboxymethyl | 336 | 22 | 37 | 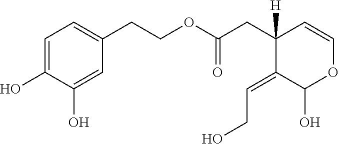 [SMILES: O=C(C[C@@H]1C=COC(O)/C1=C\CO)OCCc1ccc(O)c(O)c1] |
| 3,4-DHPEA- DETA * | 350 | 19 | 39 | 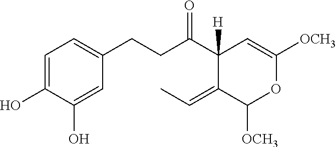 [SMILES: C/C=C1/C(OC)OC(OC)=C[C@H]1C(=O)CCc1ccc(O)c(O)c1] |
| Oleuropein aglycone (3,4- DHPEA-EA) | 378 | 31 | 32 | 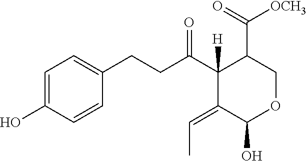 [SMILES: C/C=C1\[C@H](O)OCC(C(=O)OC)[C@H]1C(=O)CCc1ccc(O)cc1] |

Therefore, a series of phenolic compounds displaying higher affinity for COX-1 and/or COX-2 enzymes, compared to the prototypical Olea-derived compound, oleocanthal, was identified in this disclosure through docking and ADMET analysis. Among the compounds identified, 1-oleyltyrosol and ligstroside derivative show the highest affinity for COX-1 and COX-2 enzymes. However, these compounds do not meet the criteria that were set in the ADMET analysis for bioavailability. Therefore, these compounds require a vehicle, which could be a nanoparticle or microparticle formulation, for their delivery to target cells and for biological efficacy. A series of compounds that do pass the ADMET analysis and bind to COX-1 and COX-2 enzymes with higher affinity than oleocanthal, for example, methyl malate beta-hydroxytyrosol ester (Formula I, Ia-Ib, Ie-If) has been identified. These compounds are expected to possess superior biological effects than oleocanthal.

### Example 2

### Synthesis of an Exemplary Phenolic Compound (I), Methyl Malate Beta-Hydroxytyrosol Ester

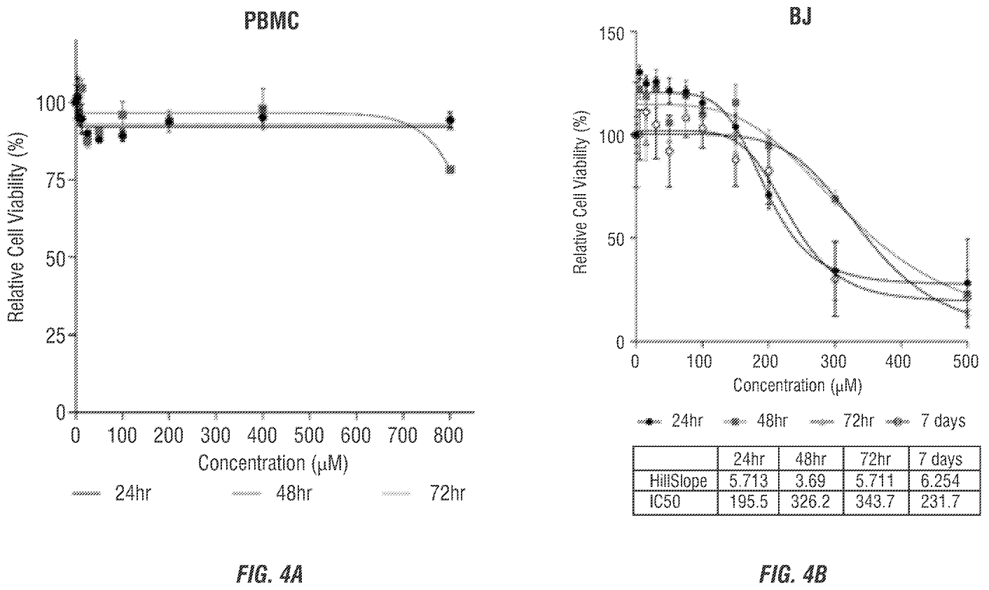

The scheme for the synthesis of the exemplary phenolic compound (I) is shown in FIG. 3. The synthesis procedure and characterization of Compound (I) is following.

### Preparation of 2-Trifluoroacetyloxysuccinic Anhydride

(R)-malic acid was placed into excess trifluoroacetic anhydride for 2 hours. The mixture was concentrated under a reduced pressure and the residue was then used directly in the next step.

### Preparation of Methyl Malic Acid

2-Trifluoroacetyloxysuccinic anhydride (60.0 g, 283 mmol) was placed into methanol (200 mL) and stirred overnight. The mixture was then concentrated under reduced pressure and recrystallized from dichloromethane (500 mL) to yield pure product, methyl malic acid (27.0 g, 64% yield). (¹H NMR (500 MHz, CDCl₃): δ=4.53 ppm (dd, J=4.2, 6.1 Hz, 1H), 3.82 ppm (s, 3H), 2.93 ppm (dd, J=4.2, 16.3 Hz, 1H), 2.85 ppm (dd, J=6.1, 16.3 Hz, 1H); ¹³C NMR (100 MHz, CDCl₃): δ=38.3 ppm, 53.0 ppm, 67.0 ppm, 173.5 ppm, 175.2 ppm).

### Preparation of TBS-Protected Methyl Malic Acid

Methyl malic acid (1.19 g, 8.04 mmol) was mixed with 50 mL of DCM and imidazole (1.64 g, 24.09 mmol). TBSOTf (4.06 mL, 17.69 mmol) was added dropwise at 0° C. The mixture was allowed to come to room temperature overnight. Methanol (10 mL) was added, the mixture was stirred for 10 minutes, and the mixture then was concentrated under reduced pressure. Methanol (100 mL) was added followed by potassium carbonate (1.3 g, 9.41 mmol) and the mixture was stirred for 2 hours. The mixture was concentrated under a reduced pressure and was taken up in ethyl acetate (100 mL), washed with 1M citric acid (50 mL), water (50 mL), and brine (50 mL). The organic layer was dried with Na₂SO₄ and concentrated under a reduced pressure. The crude mixture was purified by silica gel column chromatography (2%-5% methanol in DCM) to give the product as a colorless solid after concentration (1.04 g, 63% yield). (¹H NMR (500 MHz, CDCl₃): δ=10.57 ppm (br. s., 1H), 4.63 ppm (dd, J=4.4, 8.0 Hz, 1H), 3.74 ppm (s, 3H), 2.86 ppm (dd, J=4.4, 15.9 Hz, 1H), 2.74 ppm (dd, J=8.0, 15.9 Hz, 1H), 0.88 ppm (s, 9H), 0.11 ppm (s, 3H), 0.07 ppm (s, 3H); ¹³C NMR (100 MHz, CDCl₃): δ=−5.8 ppm, −5.0 ppm, 18.2 ppm, 25.8 ppm, 39.9 ppm, 52.2 ppm, 68.8 ppm, 172.5 ppm, 175.7 ppm).

### Preparation of Di-TBS Hydroxytyrosol

Tri-TBS-3,4-dihydroxyphenylacetic acid (2.0 g, 3.9 mmol) was placed in THF (100 mL). Lithium aluminum hydride (592 mg, 15.6 mmol) was added slowly at 0° C. The reaction was stirred for 1 hour then diluted with 200 mL of diethyl ether. Water (1 mL) was added dropwise followed by NaOH (1 mL, 15%) followed by water (3 mL). The mixture was warmed to room temperature and stirred for 15 minutes. MgSO4 was added and stirred for 15 min then filtered and concentrated under a reduced pressure. The crude material was purified by silica gel chromatography (20-30% EtOAc/Hexanes) to give Di-TBS hydroxytyrosol (1.19 g, 80% yield). (¹H NMR (400 MHz, CDCl₃): δ=6.78 ppm (d, J=8.1 Hz, 1H), 6.73-6.65 ppm (m, 2H), 3.82 ppm (t, J=6.6 Hz, 2H), 2.77 ppm (t, J=6.5 Hz, 2H), 1.01 ppm (s, 18H), 0.21 ppm (s, 12H)).

### Preparation of Tri-TBS Protected Compound (I)

TBS-methyl malic acid (1.00 g, 3.86 mmol) was added to 100 mL DCM followed by DCC (1.20 g, 5.70 mmol), Di-TBS hydroxytyrosol (1.48 g, 3.86 mmol), DMAP (140 mg, 1.14 mmol) and triethylamine (536 μL, 3.86 mmol). The mixture was stirred overnight, then washed with 50 mL water, 50 mL brine, dried with Na₂SO₄ and concentrated under a reduced pressure. The crude product was purified with silica gel column chromatography (conditions) to give the product (875 mg, 36% yield). (¹H NMR (500 MHz, CDCl₃): δ=6.81-6.59 ppm (m, 3H), 4.63 ppm (dd, J=4.9, 7.8 Hz, 1H), 4.27-4.16 ppm (m, 2H), 3.74 ppm (s, 3H), 2.85-2.75 ppm (m, 4H), 2.69 ppm (dd, J=7.8, 15.1 Hz, 1H), 0.99 ppm (s, 9H), 0.98 ppm (s, 9H), 0.88 ppm (s, 9H), 0.19 ppm (s, 6H), 0.11 ppm (s, 3H), 0.07 ppm (s, 3H)).

### Preparation of Compound (I-1)

Tetrabutyl ammonium fluoride (24 mL, 1 M in THF, 24 mmol) was added dropwise to Tri-TBS Protected Compound (I) (4.30 g, 6.86 mmol) in 70 mL THF. The mixture was stirred for 15 minutes after which TLC indicated the reaction was complete. The mixture was poured into 250 mL water and extracted with EtOAc (200×3). Organic layer was washed with brine and dried with Na₂SO₄ and was concentrated under reduced pressure to yield 3.42 g of the crude Compound (I-1). Column chromatography (from 50% EtOAc/Hexanes to 100% EtOAc) yielded 1.725 g (89% yield) of Compound (I-1) after evaporation. (¹H NMR (500 MHz, CDCl₃): δ=6.79 ppm (d, J=8.0 Hz, 1H), 6.73 ppm (d, J=1.3 Hz, 1H), 6.62 ppm (dd, J=1.3, 8.0 Hz, 1H), 5.84 ppm (br. s., 1H), 5.53 ppm (br. s., 1H), 4.51 ppm (d, J=3.9 Hz, 1H), 4.28 ppm (t, J=6.8 Hz, 2H), 3.79 ppm (s, 3H), 3.41 ppm (d, J=4.4 Hz, 1H), 2.87 ppm (dd, J=3.9, 16.6 Hz, 1H), 2.84-2.76 ppm (m, 3H)).

### Example 3

### Biological Profiling of Compound (I-1) and Other Known Compounds

### Cell Culture

Human peripheral blood mononuclear cells (PBMCs) were fractionated using the Ficoll Plaque fractionation method from whole blood obtained from the Australian Red Cross Blood Bank (ARCB) under ethic approval (#304/12). Cells were harvested fresh on the day of the experiments and maintained in complete-RPMI-1640 medium supplemented with 10% FBS, 2 mM L-glutamine and 20 μg/mL gentamicin at 37° C., 5% (v/v) CO₂. Human aortic endothelial cells (HAECs, ATCC) and the equivalent cells taken for patients with Type II diabetes (DII-HAECs, ATCC) were cultured in endothelial cell growth medium (ECM) supplemented with 100 U/ml penicillin, 100 μg/ml streptomycin, 0.25 μg/ml fungizone, 2 mM L-glutamine, 5 U/ml heparin, 30-50 μg/ml endothelial cell growth supplement (Technoclone, Vienna, Austria). All cells were passaged by typsination and seeded into 6 well culture dishes at cell densities of 1×106 cells per well; at 37° C., 5% (v/v) CO₂. Primary skin fibroblasts, BJ cells were cultured in DMEM:F12 basal medium supplemented with 2 mM L-glutamine at 37° C., 5% (v/v) CO₂.

### Cell Proliferation Studies of Compound (I-1)

Cells were seeded at densities of 7,500 cells/in black flat bottom 96-well plates (Nalge Nunc, Penfield, N.Y., USA) and treated with a dose response of methyl malate-β-hydroxytyrosol ester (MMBHTE), e.g., Compound (I) (0-800 μM) for 24, 48 or 72 hours at 37° C., 5% (v/v) CO₂. Cell viability was measured using the Cell-Titer Blue® Assay kit (Promega, Madison, Wis., USA) according to the manufacturer's instructions. Cell-Titer Blue reagent was added to each well and incubated for 4 hours at 37° C., 5% CO₂, before reading fluorescence intensity (550 nm excitation; 615 nm emission) using the CLARIOstar (BMG LABTECH, Offenburg, Germany) microplate reader.

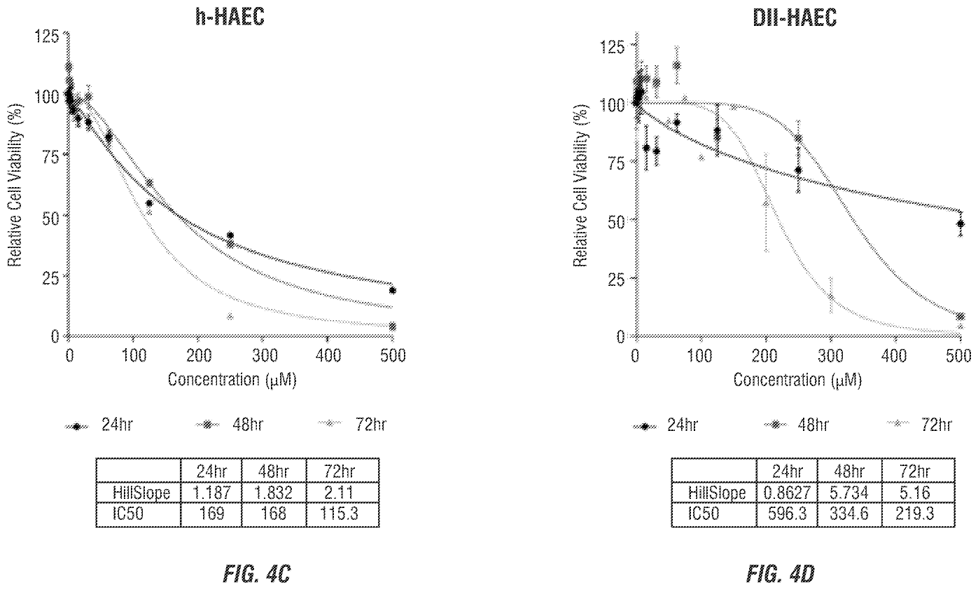

FIG. 4A, FIG. 4B, FIG. 4C, and FIG. 4D show the cell viability and proliferation of PBMC, BJ, h-HAEC, and DII-HAEC human primary cells lines treated with MMBHTE, e.g., Compound (I-1), respectively. Primary peripheral blood mononuclear cells (PBMC), primary human aortic endothelial cells taken from a healthy patient (h-HAEC) and patient with Type II diabetes (DII-HAEC) and primary skin fibroblasts (BJ) where treated with MMBHTE (0-800 μm) for up to 7 days. Shown in FIG. 4A-FIG. 4D are curves with best fit and standard error of the means from 3 independent experiments, hillslope and IC50 from the curves.

Based on these results, MMBHTE was found to have a very favorable toxicity profile, requiring relatively large doses of the compound for toxicity for the primary cell lines PBMC, h-HAEC, DII-HAEC, and BJ.

### LSD1 Demethylase Profiling of Compound (I-1) and Hydroxytyrosol (HT)

MMBHTE, e.g., Compound (I-1), and hydroxytyrosol (HT) were tested against positive control tranylcypromine (TCP) for LSD1 enzyme activity inhibition using the EpiQuik Histone Demethylase LSD1 inhibitor screening assay core kit (EpiGentek, Farmingdale, N.Y., USA) according to manufacturer's instructions. All compounds where tested in 10-dose IC₅₀ mode with 2-fold serial dilution in duplicate starting at 200 μM. The production of FAD-dependent H₂O₂ as a result of demethylase activity of LSD1 is measured by coupling with HRP and flurogen 10-Acetyl-3,7-dihydroxy Phenoxazine and fluorescence was measured using the CLARIOstar microplate reader at Ex/Em=535/590 nm. Data was represented at the percentage of LSD1 enzymatic activity inhibition relative to DMSO controls and a curve fit for the control compound.

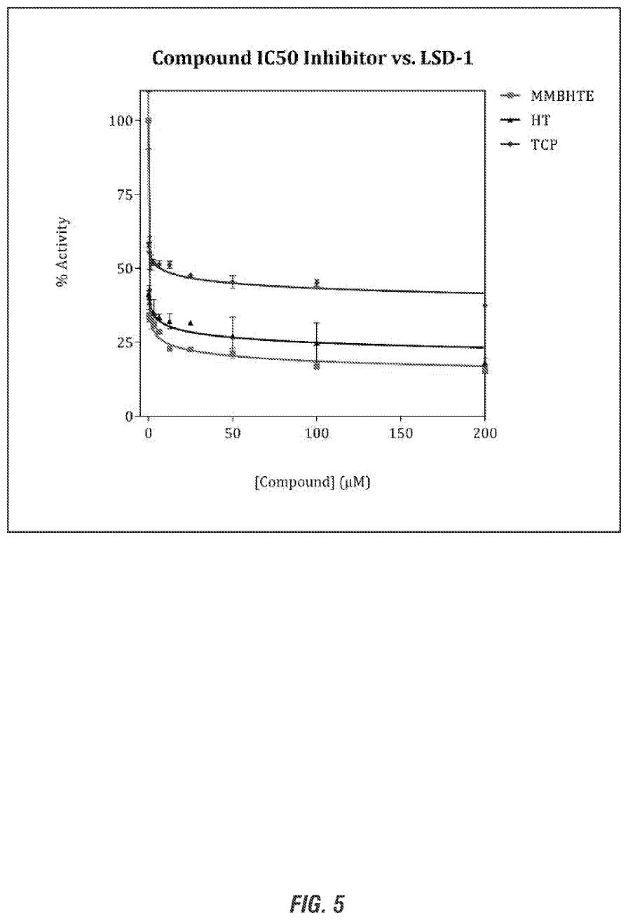

FIG. 5 shows the inhibition profiles of MMBHTE, HT, and TCP for lysine-specific demethylase 1 (LSD-1) enzyme activity. Shown in FIG. 5 are the best-fit curves for the mean dose-dependent inhibitions from 3 replicates and standard error of the mean for each compound. The IC50 values were obtained from the curves. Based on these results, HT was found to be a potent LSD-1 inhibitor (IC50 21.8 nM±2.22) in comparison to the known standard TCP (IC50 6.83 μM±2.33). More importantly, MMBHTE was found to display inhibition and specificity to the LSD1 enzyme with an IC50 of 11.89 nM±1.16.

Since the MMBHTE was found to be a better inhibitor than tranylcypromine (TCP, contracted from trans-2-phenylcyclopropylamine; original trade name Parnate) as LSD-1 inhibitor, it can be used to treat or prevent cancer.

### COX-1 and COX-2 Enzymatic Activity Profiling of Compound (I-1) and Oleocanthal

Inhibition of individual COX-1 and COX-2 enzymes by MMBHTE and oleocanthal was measured using the COX-1/2 inhibitor screening kit (BioVision, Milpatas, Calif., USA), according to the manufacturer's instructions. Both phenolic compounds were tested in 10-dose IC₅₀ mode with 2-fold serial dilution in duplicate starting at 1000 μM. The production of prostaglandin G2, the intermediate product generated by the COX enzyme in the presence of arachidonic acid is measured by coupling with HRP and COX substrate and fluorescence was measured using the CLARIOstar microplate reader at Ex/Em=535/587 nm. Data is represented at the percentage of COX enzymatic activity inhibition relative to the log of the compound and a curve fit for the control compound. Corresponding IC₅₀ values were calculated.

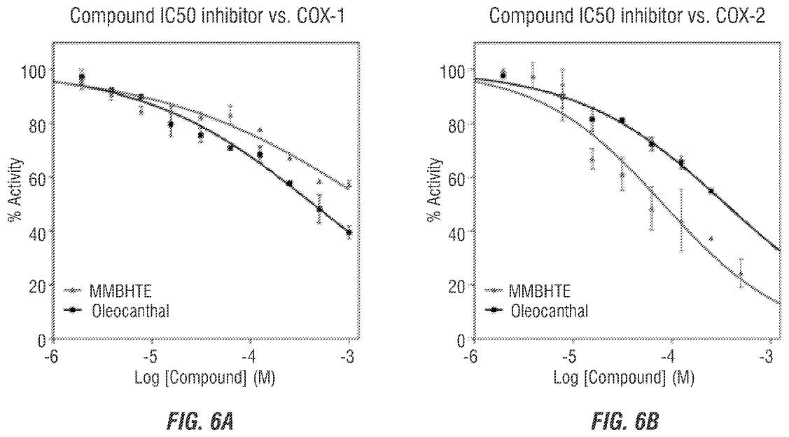

FIG. 6A and FIG. 6B show the inhibition activities of MMBHTE and Oleocanthal on COX-1 and COX-2, respectively. The data shown in FIG. 6A and FIG. 6B indicates all the test samples fell within the detectable range. The data shown in FIG. 6A and FIG. 6B is represented as the percentage of COX enzymatic activity inhibition relative to the log of the concentrations and best-fit curves for MMBHTE and Oleocanthal. Corresponding IC50 values were calculated. A dose-dependent inhibition of COX-1 or COX-2 was observed with IC50 values 1.72±0.09 and 81.6±7.7 μM for MMBHTE and 0.43±0.05 and 352.2±7.7 μM for oleocanthal respectively. Therefore, MMBHTE is a specific COX-1 and COX-2 inhibitor.

Since MMBHTE was predicted and demonstrated to behave like oleocanthal, oleuropein, or some of their derivatives, MMBHTE and other phenolic compounds disclosed herein can function as COX-1 and COX-2 inhibitor like ibuprofen and can be used as a prevention agent against or therapeutic agent for the various disorders or conditions that can be modulated by COX-1 and/or COX-2 activities.

### Monoamine Oxidase A (MAO-A) Inhibitor Profiling of Compound (I-1), HT, and TCP

Inhibitions of the MAO-A enzyme by MMBHTE, HT, and TCP were measured using the Monoamine Oxidase A (MAO-A) Inhibitor Screening Kit (Sigma-Aldrich, St Louis, Mo., USA), according to the manufacturer's instructions. The phenolic compounds were tested in 10-dose IC₅₀ mode with 2-fold serial dilution in duplicate starting at 100 μM. The production of H₂O₂ generated by the oxidative deamination of the MAO substrate tyramine was measured fluorometrically using the CLARIOstar microplate reader at Ex/Em=535/587 nm. Data is represented as the percentage of MAO enzymatic activity inhibition relative to the log of the compound and a curve fit for the control compound. Corresponding IC₅₀ values were calculated.

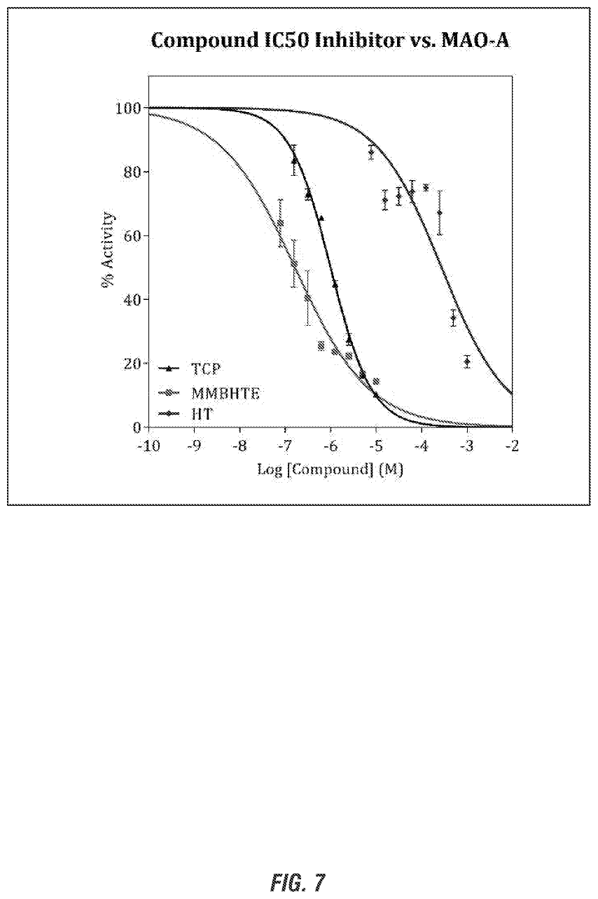

FIG. 7 shows the inhibition activities of MMBHTE, HT and TCP on monoamine oxidase A (MAO-A). Tranylcypromine (TCP) was used as a positive control compound.

The data shown in FIG. 7 are the best-fit curves of the mean percentage of MAO-A enzymatic activity inhibition from 3 independent experiments vs. the log of the concentrations compound. The data in FIG. 7 indicates that the specificity to the MAO-A enzyme was greatest for MMBHTE, with HT being lowest and TCP in the middle. The IC50 values are 0.443±0.158, 0.812±0.446, and 39.1±31.5 μM for MMBHTE, TCP, and HT respectively. The data shows that MMBHTE, e.g., Compound (I-I) is indeed a selective potent inhibitor of monoamine oxidase A (MAO-A) compared to the positive control Tranylcypromine (TCP).

Since MMBHTE was found to be a better inhibitor than tranylcypromine (TCP, contracted from trans-2-phenylcyclopropylamine; original trade name Parnate) as a nonselective and irreversible inhibitor of the enzyme monoamine oxidase, it can be used as an antidepressant and anxiolytic agent in the clinical treatment of mood and anxiety disorders, respectively. Alternatively, since olive oil was not found to interfere with SSRI, tricyclics, meperidine, tramadol, or dextromethorphan, MMBHTE can be co-administered with SSRI, tricyclics, meperidine, tramadol, or dextromethorphan.

The disclosures being thus described, it will be obvious that the same may be varied in many ways. Such variations are not to be regarded as a departure from the spirit and scope of the disclosures and all such modifications are intended to be included within the scope of the following claims.

1: A method of inhibiting COX-1, COX-2, MAO, and/or LSD-1 activity in a patient comprising:
administering to a patient an effective amount of a composition, wherein the composition comprises a compound having any one of the following formulas, stereo-isomers thereof, or salts thereof: 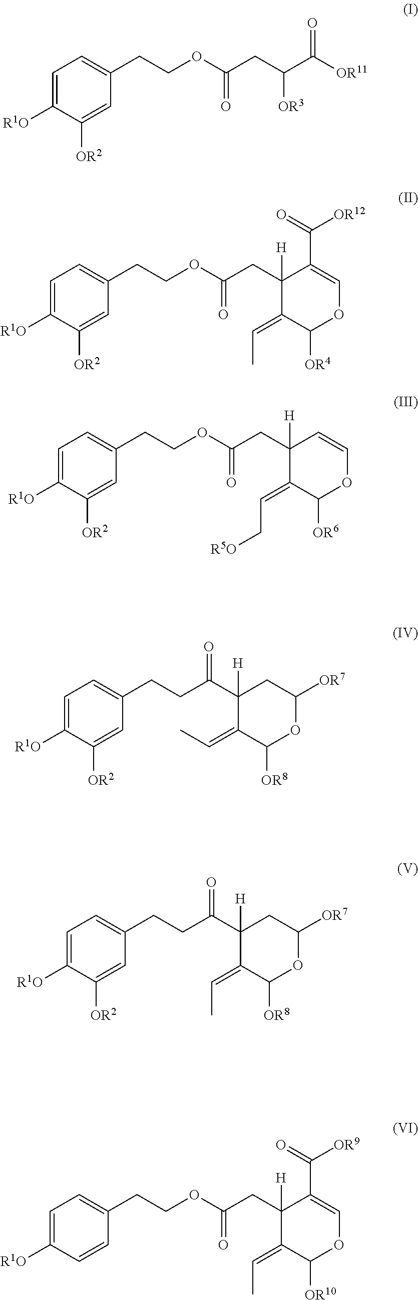
wherein R¹-R¹² are independently H or R²⁰; and R²⁰ is a H, C₁-C₅ alkyl, or a glycoside.

2: The method of claim 1, wherein the compound is one having any one of the following formulas, stereo-isomer thereof, or salt thereof: 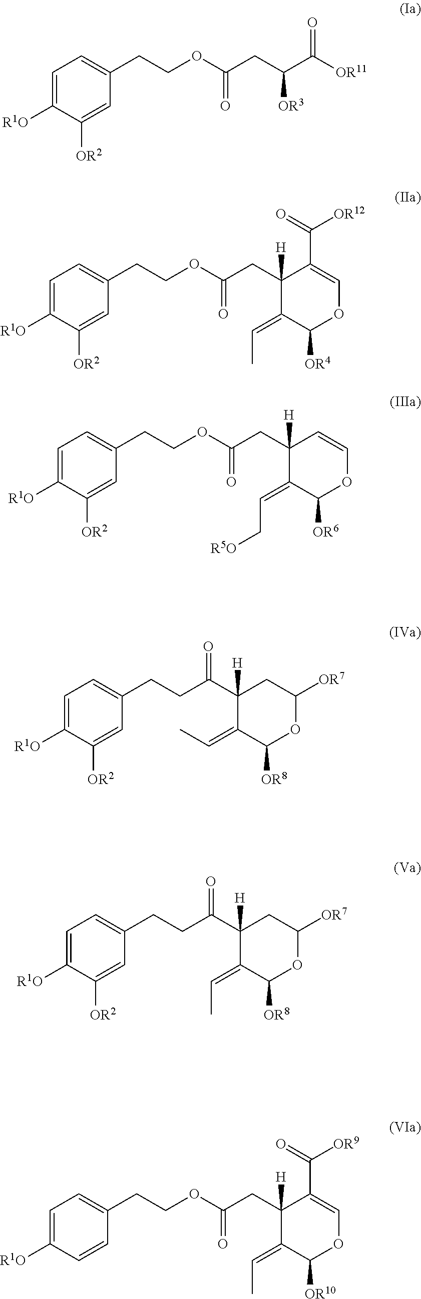
wherein R¹-R¹² are independently H or R²⁰; and R²⁰ is a H, C₁-C₅ alkyl, or a glycoside.

3: The method of claim 1, wherein the compound is one having any one of the following formulas, stereo-isomer thereof, or salt thereof: 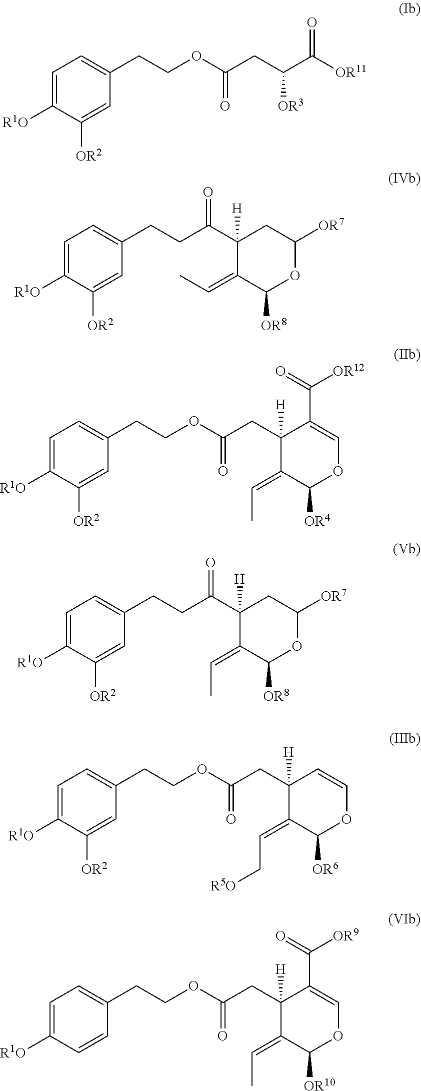
wherein R¹-R¹² are independently H or R²⁰; and R²⁰ is a H, C₁-C₅ alkyl, or a glycoside.

4: The method of claim 1, wherein the compound is one having any one of the following formulas, stereo-isomer thereof, or salt thereof. 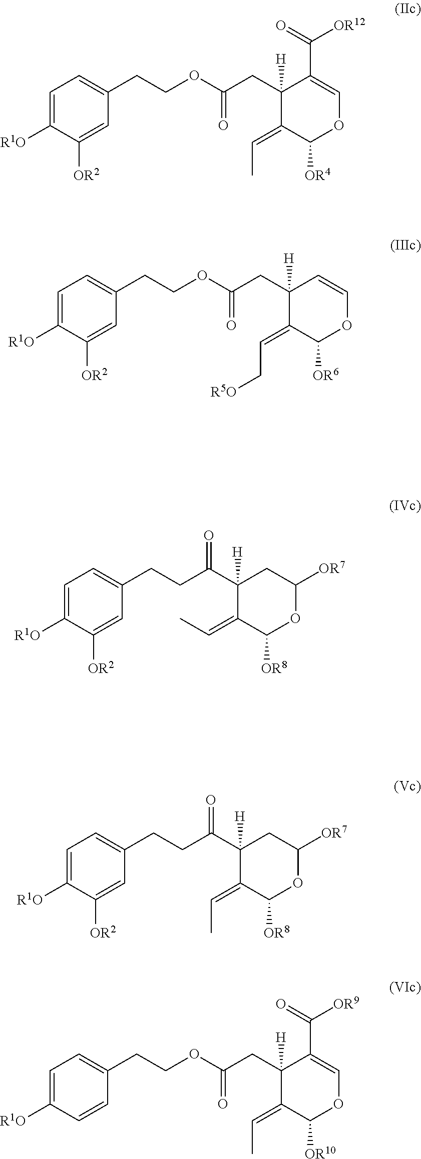
wherein R¹-R¹² are independently H or R²⁰; and R²⁰ is a H, C₁-C₅ alkyl, or a glycoside.

5: The method of claim 1, wherein the compound is one having any one of the following formulas, stereo-isomer thereof, or salt thereof: 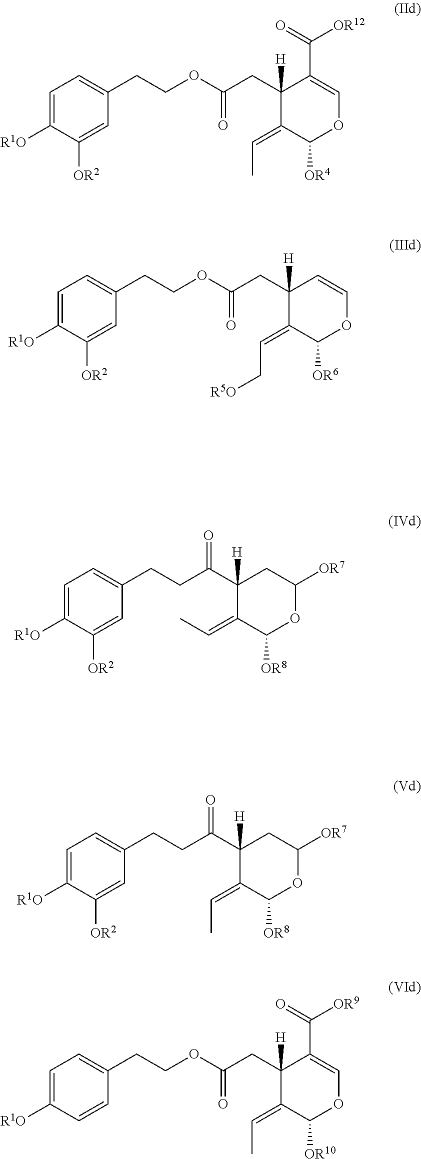
wherein R¹-R¹² are independently H or R²⁰; and R²⁰ is a H, C₁-C₅ alkyl, or a glycoside.

6: The method of claim 1, wherein the compound is one having any one of the following formulas or its isomer:

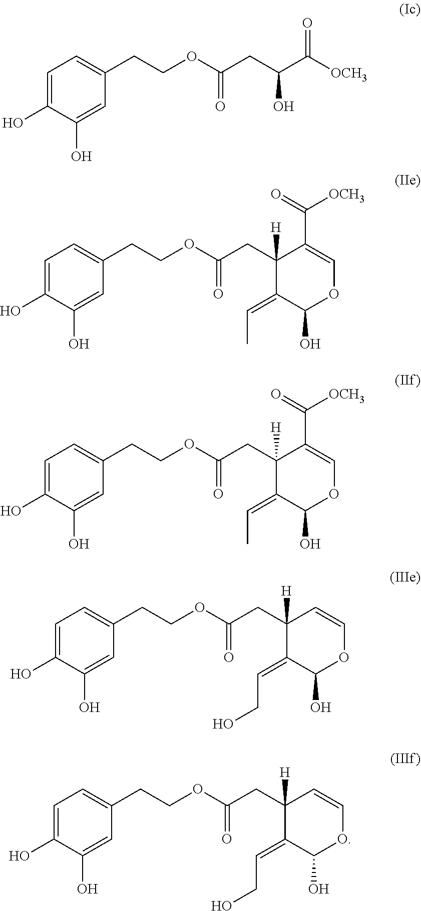 [SMILES: COC(=O)[C@@H](O)CC(=O)OCCc1ccc(O)c(O)c1 ; C/C=C1\[C@H](O)OC=C(C(=O)OC)[C@H]1CC(=O)OCCc1ccc(O)c(O)c1 ; C/C=C1\[C@H](O)OC=C(C(=O)OC)[C@@H]1CC(=O)OCCc1ccc(O)c(O)c1 ; O=C(C[C@@H]1C=CO[C@@H](O)/C1=C\CO)OCCc1ccc(O)c(O)c1 ; O=C(C[C@@H]1C=CO[C@H](O)/C1=C\CO)OCCc1ccc(O)c(O)c1]

7: The method of claim 1, wherein the compound is one having any one of the following formulas or its isomer:

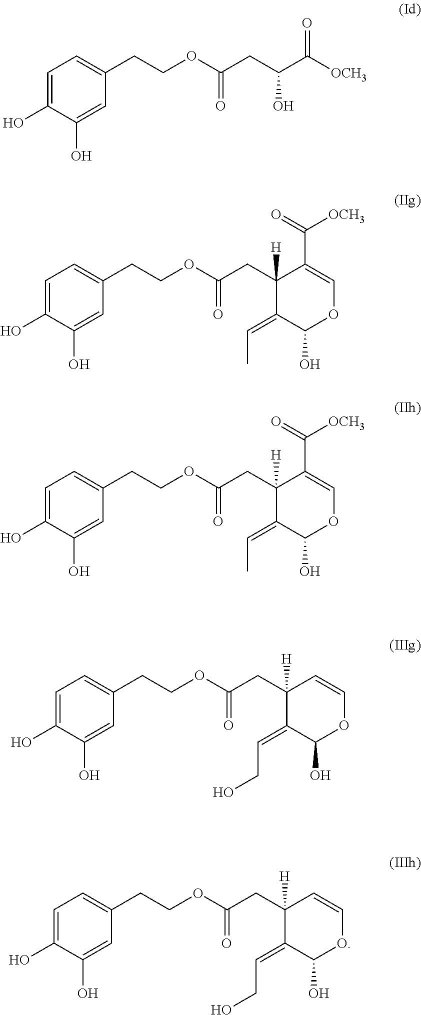 [SMILES: COC(=O)[C@H](O)CC(=O)OCCc1ccc(O)c(O)c1 ; C/C=C1/[C@H](CC(=O)OCCc2ccc(O)c(O)c2)C(C(=O)OC)=CO[C@@H]1O ; C/C=C1\[C@@H](O)OC=C(C(=O)OC)[C@@H]1CC(=O)OCCc1ccc(O)c(O)c1 ; O=C(C[C@H]1C=CO[C@@H](O)/C1=C\CO)OCCc1ccc(O)c(O)c1 ; O=C(C[C@H]1C=CO[C@H](O)/C1=C\CO)OCCc1ccc(O)c(O)c1]

8: The method of claim 1, wherein the compound is one having any one of the following formulas, stereo-isomer thereof, or salt thereof:

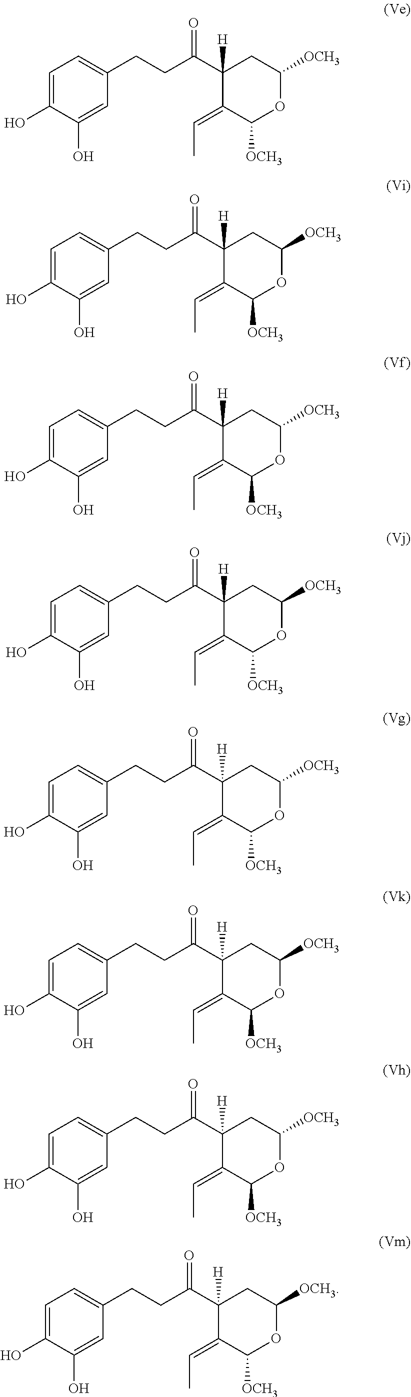

9: The method of claim 1, wherein the compound is one having any one of the following formula or its isomer:

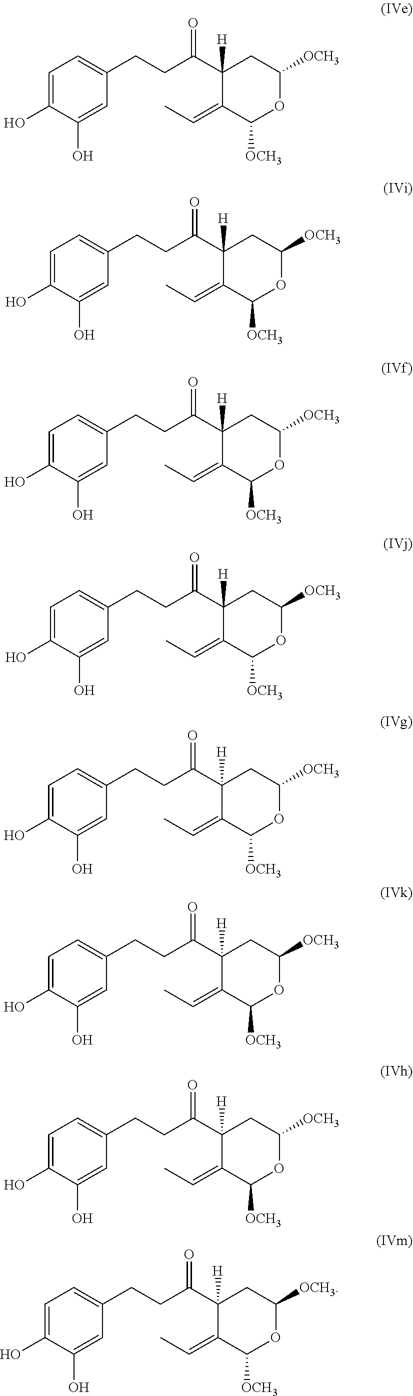

10: The method of claim 1, wherein the compound is one having any one of the following formulas, stereo-isomer thereof, or salt thereof.

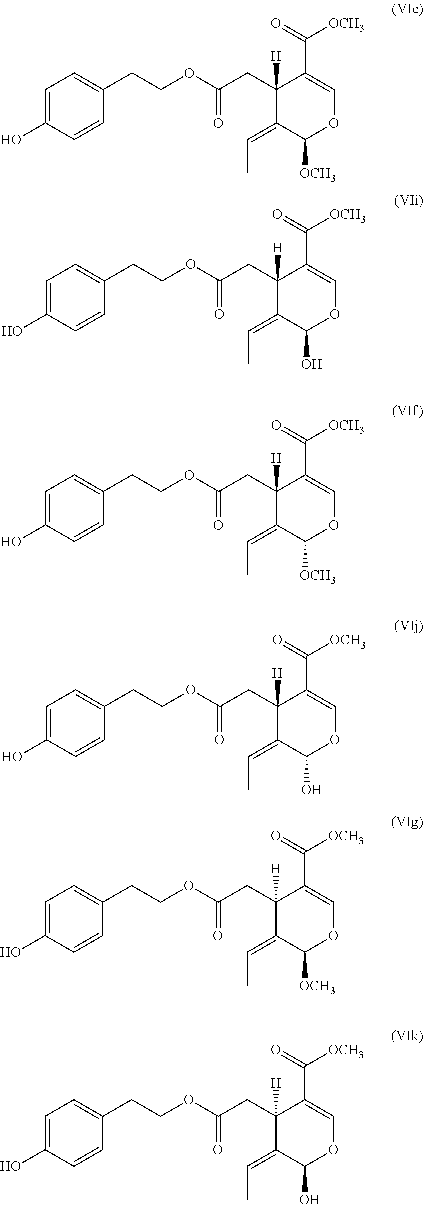 [SMILES: C/C=C1\[C@H](OC)OC=C(C(=O)OC)[C@H]1CC(=O)OCCc1ccc(O)cc1 ; C/C=C1\[C@H](O)OC=C(C(=O)OC)[C@H]1CC(=O)OCCc1ccc(O)cc1 ; C/C=C1\[C@@H](OC)OC=C(C(=O)OC)[C@H]1CC(=O)OCCc1ccc(O)cc1 ; C/C=C1/[C@H](CC(=O)OCCc2ccc(O)cc2)C(C(=O)OC)=CO[C@@H]1O ; C/C=C1\[C@H](OC)OC=C(C(=O)OC)[C@@H]1CC(=O)OCCc1ccc(O)cc1 ; C/C=C1\[C@H](O)OC=C(C(=O)OC)[C@@H]1CC(=O)OCCc1ccc(O)cc1]

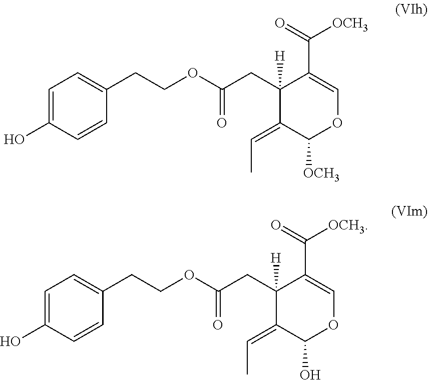 [SMILES: C/C=C1\[C@@H](OC)OC=C(C(=O)OC)[C@@H]1CC(=O)OCCc1ccc(O)cc1 ; C/C=C1\[C@@H](O)OC=C(C(=O)OC)[C@@H]1CC(=O)OCCc1ccc(O)cc1]

11: A composition comprising a therapeutically effective amount of a compound having any of the following formula, stereo-isomer thereof, or salt thereof: 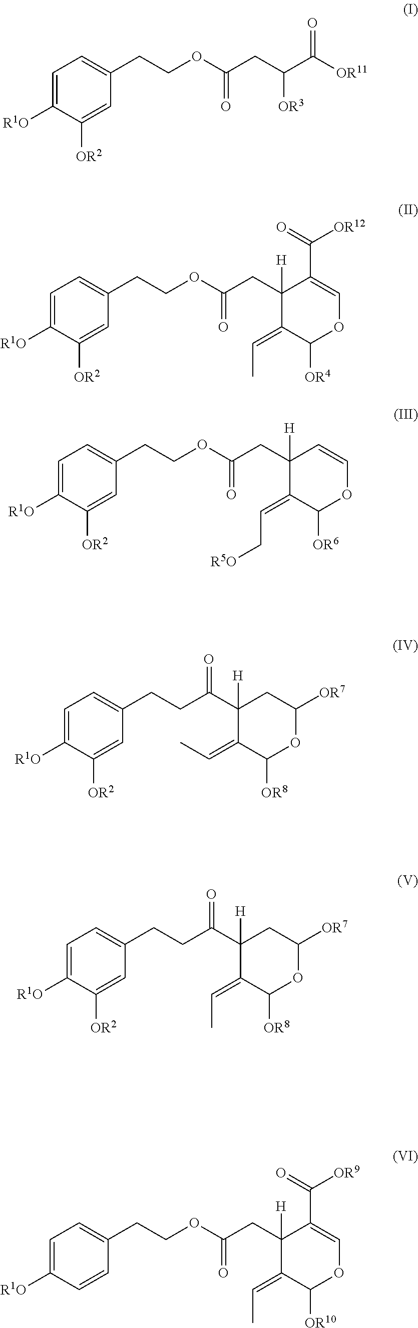
wherein R¹-R¹² are independently H or R²⁰; and R²⁰ is a H, C₁-C₅ alkyl, or a glycoside, and a pharmaceutically acceptable carrier.

12: The composition of claim 11, wherein the compound has any of the following formula, stereo-isomer thereof, or salt thereof: 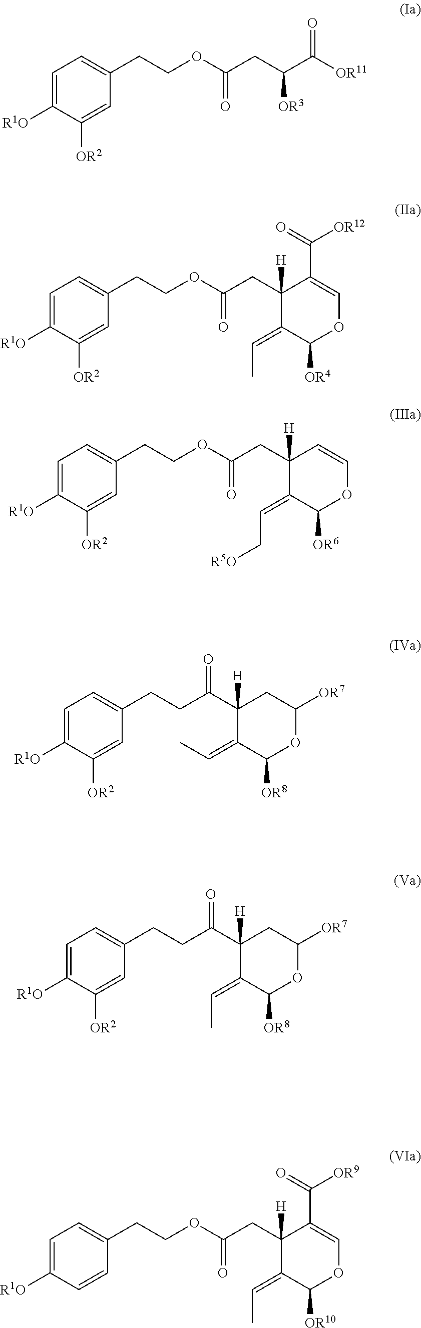
wherein R¹-R¹² are independently H or R²⁰; and R²⁰ is a H, C₁-C₅ alkyl, or a glycoside.

13: The composition of claim 11, wherein the compound has any of the following formula, stereo-isomer thereof, or salt thereof:

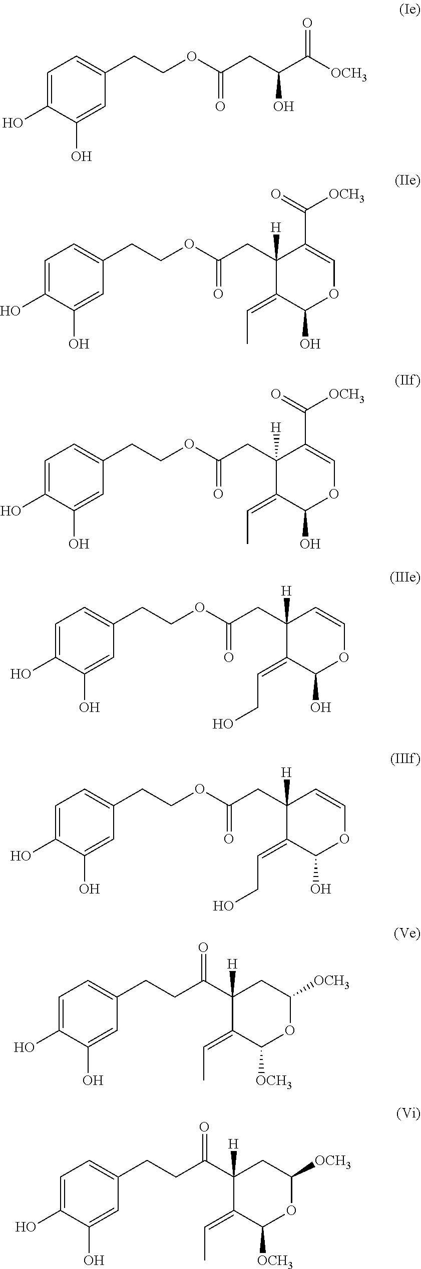

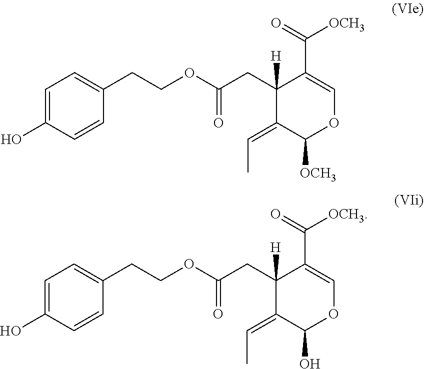 [SMILES: C/C=C1\[C@H](OC)OC=C(C(=O)OC)[C@H]1CC(=O)OCCc1ccc(O)cc1 ; C/C=C1\[C@H](O)OC=C(C(=O)OC)[C@H]1CC(=O)OCCc1ccc(O)cc1]

14: A method of treating a patient with an inflammatory disorder comprising: administering to a patient with an inflammatory disorder an effective amount of a composition comprising a compound having any of the following formula, stereo-isomer thereof, or salt thereof: 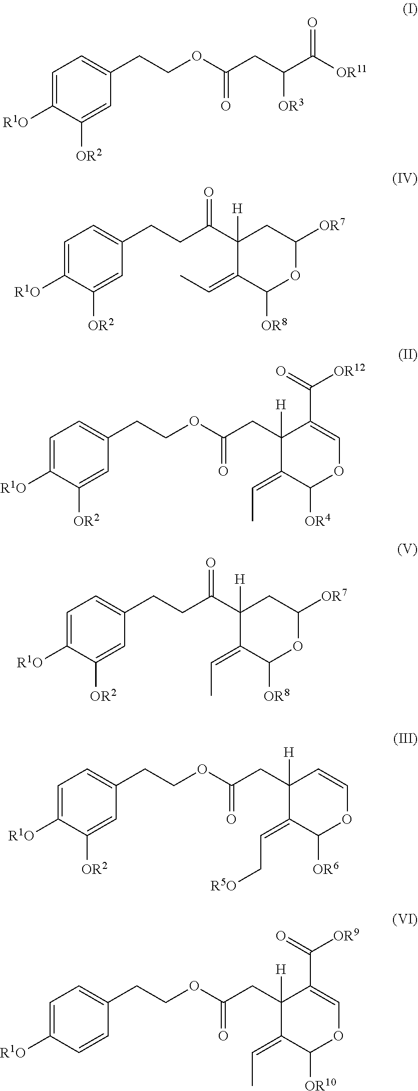
wherein R¹-R¹² are independently H or R²⁰; and R²⁰ is a H, C₁-C₅ alkyl, or a glycoside, wherein the composition inhibits COX-1, COX-2 activity, or combination thereof.

15: The method of claim 14, wherein the compound has any of the following formula, stereo-isomer thereof, or salt thereof: 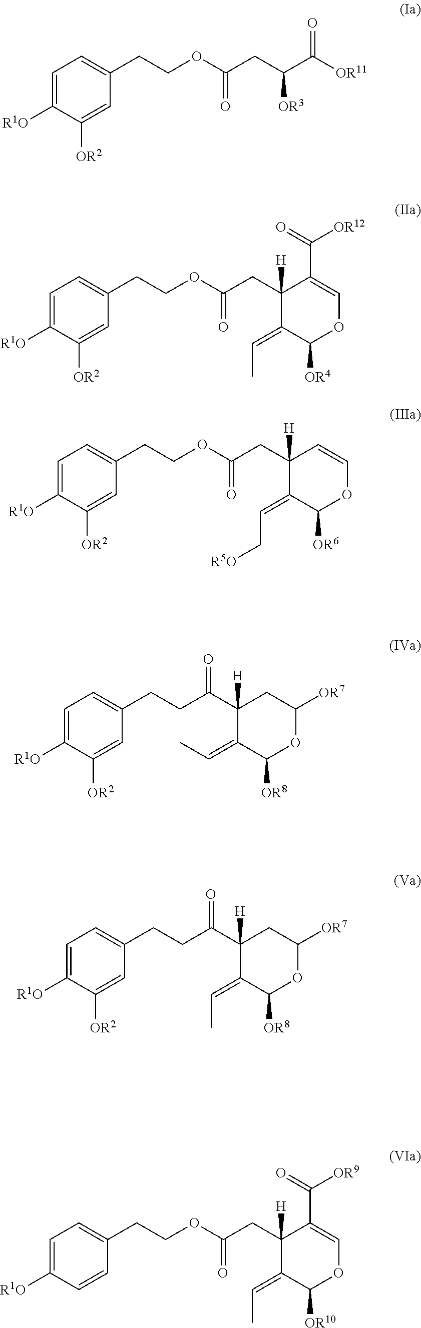
wherein R¹-R¹² are independently H or R²⁰; and R²⁰ is a H, C₁-C₅ alkyl, or a glycoside.

16: The method of claim 14, wherein the compound has any of the following formula, stereo-isomer thereof, or salt thereof:

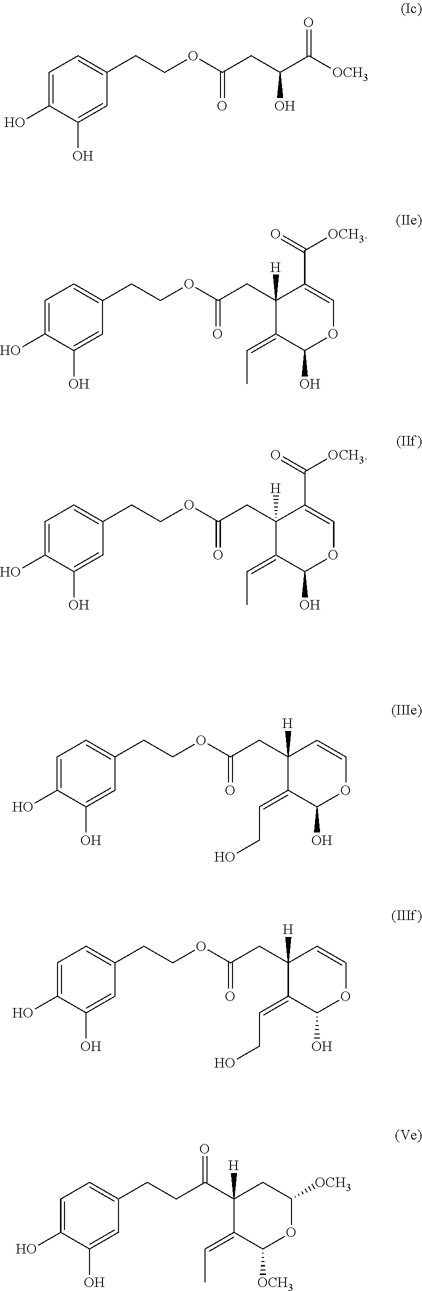 [SMILES: COC(=O)[C@@H](O)CC(=O)OCCc1ccc(O)c(O)c1 ; C/C=C1\[C@H](O)OC=C(C(=O)OC)[C@H]1CC(=O)OCCc1ccc(O)c(O)c1 ; C/C=C1\[C@H](O)OC=C(C(=O)OC)[C@@H]1CC(=O)OCCc1ccc(O)c(O)c1 ; O=C(C[C@@H]1C=CO[C@@H](O)/C1=C\CO)OCCc1ccc(O)c(O)c1 ; O=C(C[C@@H]1C=CO[C@H](O)/C1=C\CO)OCCc1ccc(O)c(O)c1 ; C/C=C1\[C@@H](OC)O[C@@H](OC)C[C@H]1C(=O)CCc1ccc(O)c(O)c1]

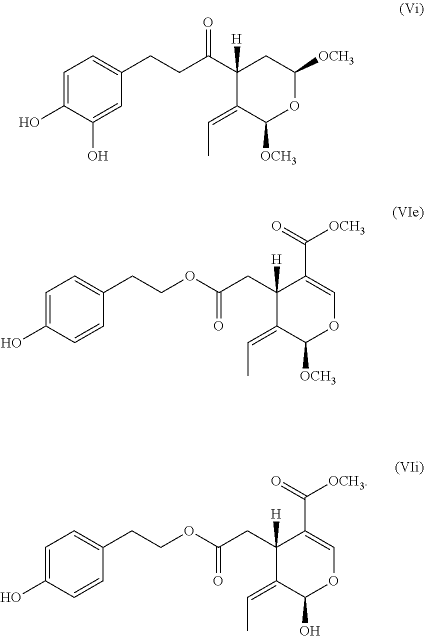 [SMILES: C/C=C1\[C@H](OC)O[C@H](OC)C[C@H]1C(=O)CCc1ccc(O)c(O)c1 ; C/C=C1\[C@H](OC)OC=C(C(=O)OC)[C@H]1CC(=O)OCCc1ccc(O)cc1 ; C/C=C1\[C@H](O)OC=C(C(=O)OC)[C@H]1CC(=O)OCCc1ccc(O)cc1]

17: The method of claim 14, wherein the compound has any of the following formula, stereo-isomer thereof, or salt thereof:

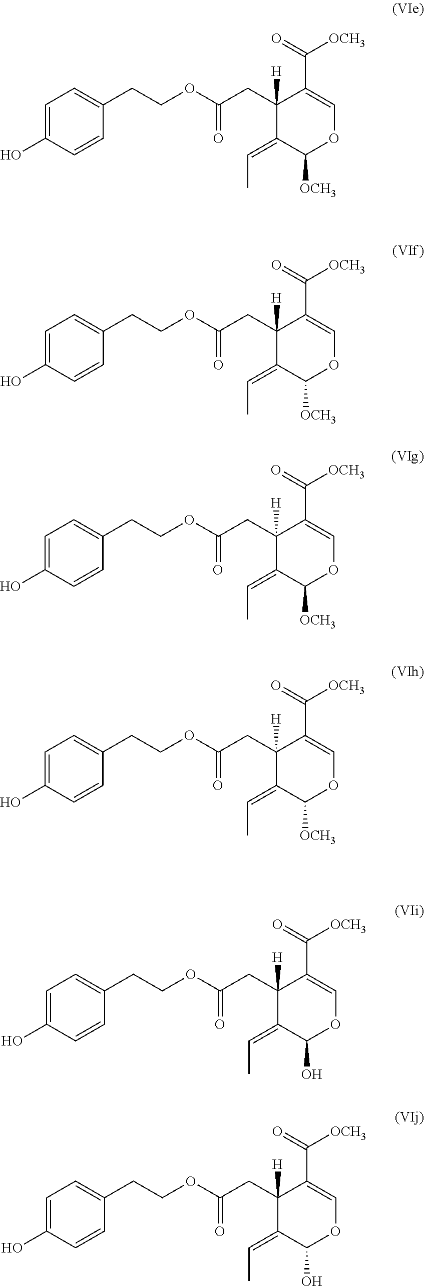 [SMILES: C/C=C1\[C@H](OC)OC=C(C(=O)OC)[C@H]1CC(=O)OCCc1ccc(O)cc1 ; C/C=C1\[C@@H](OC)OC=C(C(=O)OC)[C@H]1CC(=O)OCCc1ccc(O)cc1 ; C/C=C1\[C@H](OC)OC=C(C(=O)OC)[C@@H]1CC(=O)OCCc1ccc(O)cc1 ; C/C=C1\[C@@H](OC)OC=C(C(=O)OC)[C@@H]1CC(=O)OCCc1ccc(O)cc1 ; C/C=C1\[C@H](O)OC=C(C(=O)OC)[C@H]1CC(=O)OCCc1ccc(O)cc1 ; C/C=C1/[C@H](CC(=O)OCCc2ccc(O)cc2)C(C(=O)OC)=CO[C@@H]1O]

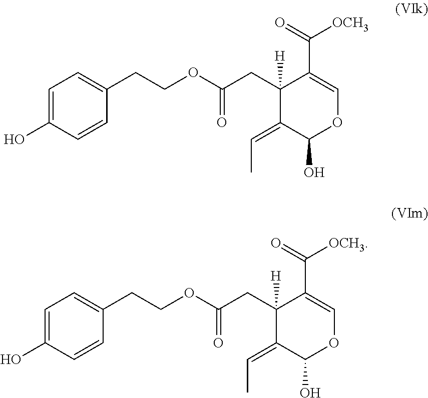 [SMILES: C/C=C1\[C@H](O)OC=C(C(=O)OC)[C@@H]1CC(=O)OCCc1ccc(O)cc1 ; C/C=C1\[C@@H](O)OC=C(C(=O)OC)[C@@H]1CC(=O)OCCc1ccc(O)cc1]

18: The method of claim 14, wherein the compound has any of formulas Ia-VIa, Ib-VIb, IIc-VIc, and IId-VId, stereo-isomer thereof, or salt thereof, wherein only one of R¹-R¹² in the molecule is methyl, C₁-C₅ alkyl, or glycoside and the rest of R¹-R¹² are H.

19: The method of claim 14, wherein said inflammatory disorder is psoriasis, cancer, asthma, allergic rhinitis, respiratory distress syndrome, inflammatory bowel disease, Chron's disease, gastritis, irritable bowel syndrome, ulcerative colitis, migraine, periarteritis nodosa, thyroiditis, aplastic anemia, Hodgkin's disease, sclerodoma, type I diabetes, myasthenia gravis, multiple sclerosis, sorcoidosis, ischemic kidney disease, nephrotic syndrome, Bechet's syndrome, polymyositis, gingivitis, conjunctivitis, vascular disease myocardial ischemia, heart disease, or stroke.

20-32. (canceled)
